SEQUENCE LISTING (22 sequences)

SEQ ID NO: 1 - protein, 11 aa, Artificial sequence
  Synthetic polypeptide
YGRKKRRQRRR

SEQ ID NO: 2 - protein, 12 aa, Artificial sequence
  Synthetic polypeptide
RRQRRTSKLMKR

SEQ ID NO: 3 - protein, 27 aa, Artificial sequence
  Synthetic polypeptide
GWTLNSAGYLLGKINLKALAALAKKIL

SEQ ID NO: 4 - protein, 33 aa, Artificial sequence
  Synthetic polypeptide
KALAWEAKLAKALAKALAKHLAKALAKALKCEA

SEQ ID NO: 5 - protein, 16 aa, Artificial sequence
  Synthetic polypeptide
RQIKIWFQNRRMKWKK

SEQ ID NO: 6 - protein, 9 aa, Artificial sequence
  Synthetic polypeptide
RKKRRQRRR

SEQ ID NO: 7 - protein, 8 aa, Artificial sequence
  Synthetic polypeptide
RKKRRQRR

SEQ ID NO: 8 - protein, 11 aa, Artificial sequence
  Synthetic polypeptide
YARAAARQARA

SEQ ID NO: 9 - protein, 11 aa, Artificial sequence
  Synthetic polypeptide
THRLPRRRRRR

SEQ ID NO: 10 - protein, 11 aa, Artificial sequence
  Synthetic polypeptide
GGRRARRRRRR

SEQ ID NO: 11 - protein, 5 aa, Artificial sequence
  Synthetic polypeptide
  MISC_FEATURE (1)..(5): This stretch of residues may be repeated 1-50 times.
GSGGS

SEQ ID NO: 12 - protein, 5 aa, Artificial sequence
  Synthetic polypeptide
  MISC_FEATURE (1)..(1): This residue may be repeated 1-20 times.
  MISC_FEATURE (1)..(5): This stretch of residues may be repeated 1-20 times.
  MISC_FEATURE (2)..(2): This residue may be repeated 1-20 times.
  MISC_FEATURE (3)..(3): This residue may be repeated 1-20 times.
  MISC_FEATURE (4)..(4): This residue may be repeated 1-20 times.
  MISC_FEATURE (5)..(5): This residue may be repeated 1-20 times.
GSGSG

SEQ ID NO: 13 - protein, 5 aa, Artificial sequence
  Synthetic polypeptide
  MISC_FEATURE (1)..(5): This stretch of residues may be repeated 1-20 times.
  MISC_FEATURE (5)..(5): This residue may be repeated 1-20 times.
GSGGS

SEQ ID NO: 14 - protein, 5 aa, Artificial sequence
  Synthetic polypeptide
  MISC_FEATURE (1)..(5): This stretch of residues may be repeated 1-20 times.
  MISC_FEATURE (4)..(4): This residue may be repeated 1-20 times.
  MISC_FEATURE (5)..(5): This residue may be repeated 1-20 times.
GSGSG

SEQ ID NO: 15 - protein, 4 aa, Artificial sequence
  Synthetic polypeptide
  MISC_FEATURE (1)..(4): This stretch of residues may be repeated 1-20 times.
  MISC_FEATURE (4)..(4): This residue may be repeated 1-20 times.
GGGS

SEQ ID NO: 16 - protein, 4 aa, Artificial sequence
  Synthetic polypeptide
GGSG

SEQ ID NO: 17 - protein, 5 aa, Artificial sequence
  Synthetic polypeptide
GGSGG

SEQ ID NO: 18 - protein, 5 aa, Artificial sequence
  Synthetic polypeptide
GSGGG

SEQ ID NO: 19 - protein, 5 aa, Artificial sequence
  Synthetic polypeptide
GGGSG

SEQ ID NO: 20 - protein, 5 aa, Artificial sequence
  Synthetic polypeptide
GSSSG

SEQ ID NO: 21 - protein, 4 aa, Artificial sequence
  Synthetic polypeptide
  MISC_FEATURE (1)..(4): This stretch of residues may be repeated 1-50 times.
GGGS

SEQ ID NO: 22 - protein, 5 aa, Artificial sequence
  Synthetic polypeptide
  MISC_FEATURE (1)..(5): This stretch of residues may be repeated 1-50 times.
GGGGS

(19) United States
(12) Patent Application Publication
(10) Pub. No.: US 2017/0362291 A1
(43) Pub. Date: Dec. 21, 2017

(54) Methods of Improving Yield in Recombinant Protein Production

(71) Applicant: ARMO BioSciences, Inc., Redwood City, CA (US)

(72) Inventors: Jackie Shek Kei Chan, Mountain View, CA (US); Brett Harold Jorgensen, Fremont, CA (US); John Brian Mumm, Los Altos Hills, CA (US)

(21) Appl. No.: 15/532,254

(22) PCT Filed: Dec. 21, 2015
(86) PCT No.: PCT/US2015/067135

§ 371 Date: Jun. 1, 2017

Related U.S. Application Data
(60) Provisional application No. 62/096,359, filed on Dec. 23, 2014.

(51) Int. Cl. C07K 14/54; C07K 1/113

(52) U.S. Cl. CPC C07K 14/5428; C07K 1/113

(57) ABSTRACT

Methods of enhancing production of cytokines such as IL-10 by, for example, optimizing refolding conditions, are described. The methods provide an efficient, cost-effective means of manufacturing IL-10 on a commercial scale.

### CROSS-REFERENCED TO RELATED APPLICATION

This application claims priority benefit of U.S. application Ser. No. 62/096,359, filed Dec. 23, 2014, which application in incorporated herein by reference in its entirety.

### TECHNICAL FIELD

This present disclosure relates to methods of enhancing large scale production of cytokines, including optimization of protein refolding.

### INTRODUCTION

Recombinant production has become invaluable to generate a significant amount of a protein of interest for therapeutic and research purposes. Commonly used protein expression systems include those derived from bacteria (e.g., E. coli and B. subtilis), yeast (e.g., S. cerevisia), baculovirus/insect (e.g., Sf9 and Sf21), and mammalian cells. Bacterial protein expression systems are advantageous in that bacteria are easy to culture, grow quickly and produce high yields of recombinant protein. However, some proteins become insoluble as inclusion bodies that are often difficult to recover without harsh denaturants and subsequent cumbersome protein-refolding procedures.

In the recombinant protein production process, parameters such as cultivation conditions, the co-expression of chaperones, and the use of folding promoting agents are frequently of tremendous import to the process. In particular, the utilization of folding promoting agents has become instrumental in yielding functional recombinant protein. Unfortunately, the techniques used to recover proteins from inclusion bodies need to be identified and optimized for each protein of interest. [See Fahnert, B., Methods in Molecular Biology, vol. 824 (“Using Folding Promoting Agents in Recombinant Protein Production: A Review” (2012)].

Predominant refolding techniques include matrix assisted refolding, dilution refolding, pressure-driven refolding, and continuous refolding. For a specific protein, refolding techniques and conditions optimized in the laboratory (e.g., the use of a histidine-tagged protein in matrix assisted refolding process) may not be useful for large scale production due to, for example, their cost and complexity. [See Jungbauer, A. and Kaar, W., J. Biotech. (“Current Status of Technical Protein Refolding” (2006)]. The inability to produce a protein in an efficient, cost effective manner might result in an otherwise useful therapeutic agent never reaching the market. Due to the tremendous importance of enhancing the yield of a therapeutic protein, including recovery of the protein from inclusion bodies, in large scale production, optimization of key parameters in the production process is invaluable.

### SUMMARY

The present disclosure contemplates methods of enhancing production of cytokines such as IL-10 and related IL-10 agents by, for example, optimizing refolding conditions. The methods provide an efficient, cost-effective means of manufacturing IL-10 on a commercial scale. Such optimally-produced IL-10 may be modified (e.g., pegylated) and used in compositions for the treatment and/or prevention of various diseases, disorders and conditions, and/or the symptoms thereof

At the most basic level, proteins are synthesized and regulated based on cellular functional needs. DNA comprises the “blueprints” for proteins and is decoded by highly regulated transcriptional processes to produce messenger RNA (mRNA). The message coded by mRNA is then translated into polypeptide chains. After translation, polypeptides are modified in various ways to complete their structure, designate their location or regulate their activity within the cell. Examples of such post-translational modifications include polypeptide folding into a globular protein with the help of chaperone proteins; modifications of the amino acids present (e.g., removal of the first methionine residue); and disulfide bridge formation or reduction.

Several mechanisms may be used to generate a significant amount of a protein of interest for, for example, therapeutic or research purposes. Chemical protein synthesis (e.g., solid-phase protein synthesis (SPPS)) produces highly pure protein but works well only for small proteins and peptides. Yield is generally low with chemical synthesis, and the method is prohibitively expensive for longer polypeptides.

In vitro (cell-free) protein expression and in vivo protein expression present alternative methods of generating proteins. Cell-free protein expression is the in vitro synthesis of protein using translation-compatible extracts of whole cells. When supplemented with cofactors, nucleotides and the specific gene template, these extracts can synthesize proteins of interest in a few hours. Although not sustainable for large scale production, cell-free protein expression systems allow for fast synthesis of recombinant proteins without the inconvenience of cell culture.

Cell-based systems are generally used for protein production, largely due to their ability to generate a high yield of the protein of interest. Traditional strategies for recombinant protein expression involve transfecting cells with a DNA vector that contains the template, then culturing the cells so that they transcribe and translate the desired protein. Typically, the cells are then lysed to extract the expressed protein for subsequent purification.

Both prokaryotic and eukaryotic in vivo protein expression systems are widely used. The selection of the system depends on the type of protein, the requirements for functional activity and the desired yield. The prokaryotic bacterium E. coli is the most frequently utilized host for protein expression due to its rapid growth, low production costs and high product yields. Often proteins are deposited as insoluble inclusion bodies that later require refolding to achieve biological activity. As a result of misfolding and aggregation, refolding is the yield-limiting step in the production of many proteins. Proteins derived from E.coli may be further modified after refolding by the covalent conjugation of poly(ethylene glycol) (PEG).

The present disclosure pertains, in part, to means for optimizing IL-10 production at a large (e.g., commercial) scale. One aspect of the present disclosure stems from the finding that IL-10 refolding is not volume-dependent (as had previously been reported) but rather is dependent on IL-10 concentration. In GMP production, reducing IL-10 concentration in refold from 0.7 mg/mL to ˜0.3 mg/mL was observed to double IL-10 yield.

In some embodiments, the concentration of unfolded IL-10 momoners in the refold buffer is about 0.01 g/mL to about 0.5 g/mL, about 0.02 g/mL to about 0.45 g/mL, about 0.03 g/mL to about 0.4 g/mL, about 0.04 g/mL to about 0.35 g/mL, about 0.05 g/mL to about 0.3 g/mL, about 0.06 g/mL to about 0.25 g/mL, about 0.07 g/mL to about 0.25 g/mL, about 0.08 g/mL to about 0.2 g/mL, about 0.09 g/mL to about 0.2 g/mL, about 0.1 g/mL to about 0.2 g/mL, or about 0.15 g/mL. In other embodiments, the concentration of unfolded IL-10 momoners in the refold buffer is greater than about 0.01 g/mL, greater than about 0.02 g/mL, greater than about 0.03 g/mL, greater than about 0.04 g/mL, greater than about 0.05 g/mL, greater than about 0.06 g/mL, greater than about 0.07 g/mL, greater than about 0.08 g/mL, greater than about 0.09 g/mL, greater than about 0.1 g/mL, greater than about 0.15 g/mL, greater than about 0.2 g/mL, greater than about 0.25 g/mL, or greater than about 0.3 g/mL. In further embodiments, the concentration of unfolded IL-10 momoners in the refold buffer is less about 0.5 g/mL, less than about 0.45 g/mL, less than about 0.4 g/mL, less than about 0.35 g/mL, less than about 0.3 g/mL, less than about 0.25 g/mL, less than about 0.2 g/mL, or less than about 0.1 g/mL. As described in the Experimental section, optimal IL-10 concentration in refold was determined to be ˜0.15 mg/mL; at a concentration above ˜0.15 mg/mL, material was lost because IL-10 aggregates and becomes insoluble precipitate.

Other aspects of the present disclosure relate to the presence and amount of arginine used in the refold process. The addition of L-Arginine to refold produced more than a two-fold greater amount of properly folded IL-10. The concentration of arginine is in the range of 0.01M-0.1 M arginine in particular aspects of the present disclosure. As described in the Experimental section, the presence of ˜0.1 M arginine in the ultrafiltration/diafiltration (UFDF) buffer (e.g., 20 mM Bis-Tris pH 6.5) was also found to be beneficial, increasing the yield by an estimated two-fold.

In some embodiments, the concentration of arginine is in the range of about 0.001 M to about 1.0 M, about 0.002 M to about 0.9 M, about 0.003 M to about 0.8 M, about 0.004 M to about 0.7 M, about 0.005 M to about 0.6 M, about 0.006 M to about 0.5 M, about 0.007 M to about 0.4 M, about 0.008 M to about 0.3 M, about 0.009 M to about 0.2 M, about 0.01 M to about 0.1 M, about 0.02 M to about 0.09 M, about 0.03 M to about 0.08 M, about 0.04 M to about 0.07 M, or about 0.05 M to about 0.06. In other embodiments, the concentration of arginine is greater than about 0.001 M, greater than about 0.002 M, greater than about 0.003 M, greater than about 0.004 M, greater than about 0.005 M, greater than about 0.006 M, greater than about 0.007 M, greater than about 0.008 M, greater than about 0.009 M, greater than about 0.01 M, greater than about 0.02 M, greater than about 0.03 M, greater than about 0.04 M, greater than about 0.05 M, greater than about 0.06 M, greater than about 0.07 M, greater than about 0.08 M, greater than about 0.09 M, greater than about 0.1 M, greater than about 0.15 M, greater than about 0.2 M, greater than about 0.3 M, greater than about 0.4 M, or greater than about 0.5 M. In still other embodiments, the concentration of arginine is less than about 1.0 M, less than about 0.9 M, less than about 0.8 M, less than about 0.7 M, less than about 0.6 M, less than about 0.5 M, less than about 0.4 M, less than about 0.3 M, less than about 0.2 M, less than about 0.15 M, less than about 0.1 M, less than about 0.095 M, less than about 0.09 M, less than about 0.08 M, less than about 0.07 M, less than about 0.06 M, less than about 0.05 M, less than about 0.04 M, less than about 0.03 M, less than about 0.02 M, or less than about 0.01 M.

Taken as a whole, the methods described herein yielded the optimal IL-10 refold conditions, wherein rHuIL-10 concentration is between 0.05 to 0.3 mg/mL, with arginine concentration between 0.01 and 0.1 M. Indeed, the presence of 0.1 M arginine in the refold buffer and in the UFDF buffer consistently increased the total refolded and recovered IL-10 by two-to-four—fold. In one embodiment, the final refold environment was optimally maintained at pH 8.3, in the presence of 20% Sucrose, 0.1M L-Arginine, 50 mM Tris, 0.45 mM oxidized glutathione and 0.05 mM reduced glutathione.

In particular embodiments, the present disclosure contemplates methods of generating refolded IL-10, comprising: (a) obtaining a mixture comprising unfolded IL-10 monomers, and (b) contacting the mixture with a refold buffer to produce an admixture comprising refolded IL-10; wherein the concentration of unfolded IL-10 monomers in the refold buffer is 0.05 g/mL to 0.3 g/mL. In some embodiments, the concentration of unfolded IL-10 monomers in the refold buffer is 0.1 g/mL to 0.25 g/mL, 0.1 g/mL to 0.2 g/mL, or about 0.15 g/mL. The IL-10 is recombinantly-produced human IL-10 (rhIL-10) in certain embodiments. The rhIL-10 can be expressed in bacteria (e.g., E. coli). In some embodiments, the aforementioned mixture is produced by combining a plurality of inclusion bodies comprising IL-10 with a suspension buffer. Additional embodiments further comprise adding a redox system to the refold buffer, such as a redox system that comprises oxidized and reduced glutathione.

The present disclosure contemplates embodiments wherein at least one naturally occurring or non-naturally occurring amino acid is added to the refold buffer. In some embodiments, the amino acid is arginine. In certain embodiments, 0.005 to 0.3 M arginine is added to the refold buffer, 0.0075 to 0.25 M arginine is added to the refold buffer, 0.05 M to 0.2 M arginine is added to the refold buffer, or 0.01 M to 0.15 M arginine is added to the refold buffer. In additional embodiments, the present disclosure contemplates the addition of about 0.1 M arginine and about 0.15 g/mL of unfolded IL-10 monomers to the refold buffer.

In certain embodiments, it is contemplated that a wash clarification is performed on the aforementioned mixture prior to the step of contacting the mixture with a refold buffer to produce an admixture comprising refolded IL-10. The present disclosure contemplates embodiments wherein an ultrafiltration/diafiltration (UFDF) is performed on the admixture.

The present disclosure contemplates a refold buffer pH of any value conducive to practicing the disclosures set forth herein. In certain embodiments, the pH may be less than about 7.5, about 7.5, about 7.6, about 7.7, about 7.8, about 7.9, about 8.0, about 8.1, about 8.2, about 8.3, about 8.4, about 8.5, about 8.6, about 8.7, about 8.8 or greater than about 8.9. In particular embodiments, the pH of the refold buffer is about pH 8.3.

The present disclosure also contemplates an IL-10 refold buffer, comprising: (a) a mixture comprising unfolded IL-10 monomers in a concentration of from 0.05 g/mL to 0.3 g/mL; and (b) arginine in a molarity of from 0.005 to 0.3 M. In some embodiments, the refold buffer comprises 0.0075 to 0.25 M arginine, 0.05 to 0.2 M arginine, or about 0.01 to 0.15 M arginine. Other possible concentrations of arginine are disclosed herein.

In certain embodiments, the unfolded IL-10 monomers are present in a concentration of from about 0.001 g/mL to about 1.0 g/mL, from about 0.0025 g/mL to about 0.9 g/mL, from about 0.005 g/mL to about 0.8 g/mL, from about 0.0075 g/mL to about 0.7 g/mL, from about 0.01 g/mL to about 0.6 g/mL, from about 0.02 g/mL to about 0.5 g/mL, from about 0.03 g/mL to about 0.4 g/mL, from about 0.04 g/mL to about 0.35 g/mL, or from about 0.05 to about 0.3 g/mL. In still further embodiments, the concentration of unfolded IL-10 monomers is from about 0.05 g/mL to about 0.25 g/mL, from about 0.1 g/mL to about 0.2 g/mL, or about 0.15 g/mL. In particular embodiments, the concentration of unfolded IL-10 monomers is from about 0.05 g/mL to about 0.25 g/mL, from about 0.1 g/mL to about 0.2 g/mL, or about 0.15 g/mL.

The present disclosure contemplates embodiments wherein the unfolded IL-10 monomers are present in a concentration greater than about 0.001 g/mL, greater than about 0.0025 g/mL, greater than about 0.005 g/mL, greater than about 0.0075 g/mL, greater than about 0.01 g/mL, greater than about 0.02 g/mL, greater than about 0.03 g/mL, greater than about 0.04 g/mL, or greater than about 0.05 g/mL. In some aspects, the present disclosures contemplates embodiments wherein the unfolded IL-10 monomers are present in a concentration less than about 1.0 g/mL, less than about 0.9 g/mL, less than about 0.8 g/mL, less than about 0.7 g/mL, less than about 0.6 g/mL, less than about 0.5 g/mL, less than about 0.4 g/mL, less than about 0.35 g/mL, less than about 0.3 g/mL, less than about 0.25 g/mL, less than about 0.2 g/mL, less than about 0.15 g/mL or less than about 0.1 g/mL.

A particular embodiment contemplates a refold buffer comprising about 0.1M arginine and about 0.15 g/mL of unfolded IL-10 monomers.

As discussed further hereafter, human IL-10 is a homodimer and each monomer comprises 178 amino acids, the first 18 of which comprise a signal peptide. Particular embodiments of the present disclosure comprise mature human IL-10 polypeptides lacking the signal peptide (see, e.g., U.S. Pat. No. 6,217,857). In further particular embodiments, the IL-10 agent is a variant of mature human IL-10. The variant may exhibit activity less than, comparable to, or greater than the activity of mature human IL-10; in certain embodiments the activity is comparable to or greater than the activity of mature human IL-10.

The terms “IL-10”, “IL-10 polypeptide(s),” “agent(s)” and the like are intended to be construed broadly and include, for example, human and non-human IL-10—related polypeptides, including homologs, variants (including muteins), and fragments thereof, as well as IL-10 polypeptides having, for example, a leader sequence (e.g., the signal peptide), and modified versions of the foregoing. In further particular embodiments, the terms “IL-10”, “IL-10 polypeptide(s), “agent(s)” are agonists. Particular embodiments relate to pegylated IL-10, which is also referred to herein as “PEG-IL-10”.

The IL-10 agents described in the present disclosure may comprise at least one modification to form a modified IL-10 agent, wherein the modification does not alter the amino acid sequence of the IL-10 agent. Certain embodiments of the present disclosure contemplate such modifications in order to enhance one or more properties (e.g., pharmacokinetic parameters, efficacy, etc.). In some embodiments, the modified IL-10 agent is a PEG-IL-10 agent. The PEG-IL-10 agent may comprise at least one PEG molecule covalently attached to at least one amino acid residue of at least one subunit of IL-10 or comprise a mixture of mono-pegylated and di-pegylated IL-10 in other embodiments. The PEG component of the PEG-IL-10 agent may have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa. In particular embodiments, the modifications described above are site-specific, and in still others it comprises a linker.

The present disclosure contemplates pharmaceutical compositions comprising a pharmaceutically effective amount of one or more of the aforementioned agents and a pharmaceutically acceptable diluent, carrier or excipient. Generally, such compositions are suitable for human administration. These pharmaceutical compositions may comprise one or more additional prophylactic or therapeutic agents, examples of which are described herein.

The present disclosure also contemplates methods of treating or preventing an IL-10—related disease, disorder or condition in a subject (e.g., a human), comprising administering (e.g., parenterally, including subcutaneously) to the subject a therapeutically effective amount of an IL-10 agent.

Other embodiments of the present disclosure are described herein, while still others would be envisaged by the skilled artisan after reviewing this disclosure.

### DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

The publications discussed herein are provided solely for their disclosure prior to the filing date of the present application. Further, the dates of publication provided may be different from the actual publication dates, which may need to be independently confirmed.

### Overview

The present disclosure contemplates methods of enhancing large scale (e.g., commercial) production of cytokines (e.g., IL-10), including optimization of protein refolding. The cytokines (e.g., IL-10) find use in the treatment and/or prevention of a broad range of diseases, disorders and conditions, and/or the symptoms thereof, including cancer and immune-, inflammatory- and viral-related disorders.

Some of the embodiments and descriptions set forth herein are described in the context of an IL-10 agent (e.g., a PEG-IL-10 agent). It is to be understood that, when appropriate in view of the context in which it is being used, recitation of an IL-10 agent may also refer more broadly to a cytokine agent.

It should be noted that any reference to “human” in connection with the polypeptides and nucleic acid molecules of the present disclosure is not meant to be limiting with respect to the manner in which the polypeptide or nucleic acid is obtained or the source, but rather is only with reference to the sequence as it may correspond to a sequence of a naturally occurring human polypeptide or nucleic acid molecule. In addition to the human polypeptides and the nucleic acid molecules which encode them, the present disclosure contemplates IL-10—related polypeptides and corresponding nucleic acid molecules (and, in certain instances, cytokine polypeptides and corresponding nucleic acid molecules) from other species.

### Definitions

Unless otherwise indicated, the following terms are intended to have the meaning set forth below. Other terms are defined elsewhere throughout the specification.

The terms “patient” or “subject” are used interchangeably to refer to a human or a non-human animal (e.g., a mammal).

The terms “administration”, “administer” and the like, as they apply to, for example, a subject, cell, tissue, organ, or biological fluid, refer to contact of, for example, IL-10 or PEG-IL-10), a nucleic acid (e.g., a nucleic acid encoding native human IL-10); a pharmaceutical composition comprising the foregoing, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, treatment” and the like refer to a course of action (such as administering IL-10 or a pharmaceutical composition comprising IL-10) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease. The terms may also be used in other contexts, such as situations where IL-10 or PEG-IL-10 contacts an IL-10 receptor in, for example, the fluid phase or colloidal phase.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent”, “preventing”, “prevention” and the like refer to a course of action (such as administering IL-10 or a pharmaceutical composition comprising IL-10) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other caregiver that a subject requires or will benefit from preventative care. This judgment is made based on a variety of factors that are in the realm of a physician's or caregiver's expertise.

The phrase “therapeutically effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the amount of inflammatory cytokines produced following administration may be indicative of whether a therapeutically effective amount has been used.

The phrase “in a sufficient amount to effect a change” means that there is a detectable difference between a level of an indicator measured before (e.g., a baseline level) and after administration of a particular therapy. Indicators include any objective parameter (e.g., serum concentration of IL-10) or subjective parameter (e.g., a subject's feeling of well-being).

The term “small molecules” refers to chemical compounds having a molecular weight that is less than about 10 kDa, less than about 2 kDa, or less than about lkDa. Small molecules include, but are not limited to, inorganic molecules, organic molecules, organic molecules containing an inorganic component, molecules comprising a radioactive atom, and synthetic molecules. Therapeutically, a small molecule may be more permeable to cells, less susceptible to degradation, and less likely to elicit an immune response than large molecules.

The term “ligand” refers to, for example, a peptide, a polypeptide, a membrane-associated or membrane-bound molecule, or a complex thereof, that can act as an agonist or antagonist of a receptor. “Ligand” encompasses natural and synthetic ligands, e.g., cytokines, cytokine variants, analogs, muteins, and binding compositions derived from antibodies. “Ligand” also encompasses small molecules, e.g., peptide mimetics of cytokines and peptide mimetics of antibodies. The term also encompasses an agent that is neither an agonist nor antagonist, but that can bind to a receptor without significantly influencing its biological properties (e.g., signaling or adhesion). Moreover, the term includes a membrane-bound ligand that has been changed, e.g., by chemical or recombinant methods, to a soluble version of the membrane-bound ligand. A ligand or receptor may be entirely intracellular, that is, it may reside in the cytosol, nucleus, or some other intracellular compartment. The complex of a ligand and receptor is termed a “ligand-receptor complex”.

The terms “inhibitors” and “antagonists”, or “activators” and “agonists” refer to inhibitory or activating molecules, respectively, for example, for the activation of, e.g., a ligand, receptor, cofactor, gene, cell, tissue, or organ. Inhibitors are molecules that decrease, block, prevent, delay activation, inactivate, desensitize, or down-regulate, e.g., a gene, protein, ligand, receptor, or cell. Activators are molecules that increase, activate, facilitate, enhance activation, sensitize, or up-regulate, e.g., a gene, protein, ligand, receptor, or cell. An inhibitor may also be defined as a molecule that reduces, blocks, or inactivates a constitutive activity. An “agonist” is a molecule that interacts with a target to cause or promote an increase in the activation of the target. An “antagonist” is a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist.

The terms “modulate”, “modulation” and the like refer to the ability of a molecule (e.g., an activator or an inhibitor) to increase or decrease the function or activity of an agent (e.g., an IL-10 agent) (or the nucleic acid molecules encoding them), either directly or indirectly; or to enhance the ability of a molecule to produce an effect comparable to that of an agent (e.g., an IL-10 agent). The term “modulator” is meant to refer broadly to molecules that can effect the activities described above. By way of example, a modulator of, e.g., a gene, a receptor, a ligand, or a cell, is a molecule that alters an activity of the gene, receptor, ligand, or cell, where activity can be activated, inhibited, or altered in its regulatory properties. A modulator may act alone, or it may use a cofactor, e.g., a protein, metal ion, or small molecule. The term “modulator” includes agents that operate through the same mechanism of action as an agent (e.g., an IL-10 agent) (i.e., agents that modulate the same signaling pathway as an agent (e.g., an IL-10 agent) in a manner analogous thereto) and are capable of eliciting a biological response comparable to (or greater than) that of an agent (e.g., an IL-10 agent).

Examples of modulators include small molecule compounds and other bioorganic molecules. Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of an Activator.

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term may also refer to activity in modulating or maintaining cell-to-cell interactions (e.g., adhesion), or activity in maintaining a structure of a cell (e.g., a cell membrane). “Activity” can also mean specific activity, e.g., [catalytic activity]/[mg protein], or [immunological activity]/[mg protein], concentration in a biological compartment, or the like. The term “proliferative activity” encompasses an activity that promotes, that is necessary for, or that is specifically associated with, for example, normal cell division, as well as cancer, tumors, dysplasia, cell transformation, metastasis, and angiogenesis.

As used herein, “comparable”, “comparable activity”, “activity comparable to”, “comparable effect”, “effect comparable to”, and the like are relative terms that can be viewed quantitatively and/or qualitatively. The meaning of the terms is frequently dependent on the context in which they are used. By way of example, two agents that both activate a receptor can be viewed as having a comparable effect from a qualitative perspective, but the two agents can be viewed as lacking a comparable effect from a quantitative perspective if one agent is only able to achieve 20% of the activity of the other agent as determined in an art-accepted assay (e.g., a dose-response assay) or in an art-accepted animal model. When comparing one result to another result (e.g., one result to a reference standard), “comparable” frequently means that one result deviates from a reference standard by less than 35%, by less than 30%, by less than 25%, by less than 20%, by less than 15%, by less than 10%, by less than 7%, by less than 5%, by less than 4%, by less than 3%, by less than 2%, or by less than 1%. In particular embodiments, one result is comparable to a reference standard if it deviates by less than 15%, by less than 10%, or by less than 5% from the reference standard. By way of example, but not limitation, the activity or effect may refer to efficacy, stability, solubility, or immunogenicity.

The term “response,” for example, of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; and the like.

It will be appreciated that throughout this disclosure reference is made to amino acids according to the single letter or three letter codes. For the reader's convenience, the single and three letter amino acid codes are provided below:

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

As used herein, the term “variant” encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Non-naturally-occurring variants include polypeptides and nucleic acids that comprise a change in amino acid or nucleotide sequence, respectively, where the change in sequence is artificially introduced (e.g., muteins); for example, the change is generated in the laboratory by human intervention (“hand of man”). Thus, herein a “mutein” refers broadly to mutated recombinant proteins that usually carry single or multiple amino acid substitutions and are frequently derived from cloned genes that have been subjected to site-directed or random mutagenesis, or from completely synthetic genes.

The terms “DNA”, “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

“Derived from”, in the context of an amino acid sequence or polynucleotide sequence (e.g., an amino acid sequence “derived from” an IL-10 polypeptide), is meant to indicate that the polypeptide or nucleic acid has a sequence that is based on that of a reference polypeptide or nucleic acid (e.g., a naturally occurring IL-10 polypeptide or an IL-10-encoding nucleic acid), and is not meant to be limiting as to the source or method in which the protein or nucleic acid is made. By way of example, the term “derived from” includes homologs or variants of reference amino acid or DNA sequences.

In the context of a polypeptide, the term “isolated” refers to a polypeptide of interest that, if naturally occurring, is in an environment different from that in which it may naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was made by either synthetic or recombinant means.

“Enriched” means that a sample is non-naturally manipulated (e.g., by a scientist) so that a polypeptide of interest is present in a) a greater concentration (e.g., at least 3-fold greater, at least 4-fold greater, at least 8-fold greater, at least 64-fold greater, or more) than the concentration of the polypeptide in the starting sample, such as a biological sample (e.g., a sample in which the polypeptide naturally occurs or in which it is present after administration), orb) a concentration greater than that of the environment in which the polypeptide was made (e.g., as in a bacterial cell).

“Substantially pure” indicates that a component (e.g., a polypeptide) makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

The terms “specifically binds” or “selectively binds”, when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two-fold greater, at least ten times greater, at least 20-times greater, or at least 100-times greater than the affinity with any other antibody, or binding composition derived therefrom. In a particular embodiment, the antibody will have an affinity that is greater than about 10⁹ liters/mol, as determined by, e.g., Scatchard analysis (Munsen, et al. 1980 Analyt. Biochem. 107:220-239).

### IL-10 and PEG-IL-10

The anti-inflammatory cytokine IL-10, also known as human cytokine synthesis inhibitory factor (CSIF), is classified as a type(class)-2 cytokine, a set of cytokines that includes IL-19, IL-20, IL-22, IL-24 (Mda-7), and IL-26, interferons (IFN-α, -β, -γ, -δ, -ε, -κ, -Ω, and -τ) and interferon-like molecules (limitin, IL-28A, IL-28B, and IL-29).

IL-10 is a cytokine with pleiotropic effects in immunoregulation and inflammation. Although predominantly expressed in macrophages, IL-10 expression has also been detected in activated T cells, B cells, mast cells, and monocytes. It is produced by mast cells, counteracting the inflammatory effect that these cells have at the site of an allergic reaction. While IL-10 predominantly limits the production and secretion of pro-inflammatory cytokines in response to toll-like receptor agonists, it is also stimulatory towards certain T cells and mast cells and stimulates B-cell maturation, proliferation and antibody production. IL-10 can block NF-κB activity and is involved in the regulation of the JAK-STAT signaling pathway. It also induces the cytotoxic activity of CD8+ T-cells and the antibody production of B-cells, and it suppresses macrophage activity and tumor-promoting inflammation. The regulation of CD8+ T-cells is dose-dependent, wherein higher doses induce stronger cytotoxic responses.

As a result of its pleiotropic activity, IL-10 has been linked to a broad range of diseases, disorders and conditions, including inflammatory conditions, immune-related disorders, fibrotic disorders, metabolic disorders, including regulation of cholesterol, and cancer. Clinical and pre-clinical evaluations with IL-10 for a number of such diseases, disorders and conditions have solidified its therapeutic potential.

Human IL-10 is a homodimer with a molecular mass of 37 kDa, wherein each 18.5 kDa monomer comprises 178 amino acids, the first 18 of which comprise a signal peptide. Each monomer comprises four cysteine residues that form two intramolecular disulfide bonds. The IL-10 dimer becomes biologically inactive upon disruption of the non-covalent interactions between the two monomer subunits. Data obtained from the published crystal structure of IL-10 indicates that the functional dimer exhibits certain similarities to IFN-γ (Zdanov et al, (1995) Structure (Lond) 3:591-601). The description herein generally refers to the homodimer; however, certain aspects of the discussion can also apply to a monomer, as will be apparent from the context.

The various embodiments of the present disclosure contemplate human IL-10 (NP_000563) and murine IL-10 (NP_034678), which exhibit 80% homology, and use thereof. In addition, the scope of the present disclosure includes IL-10 orthologs, and modified forms thereof, from other mammalian species, including rat (accession NP_036986.2; GI 148747382); cow (accession NP_776513.1; GI 41386772); sheep (accession NP_001009327.1; GI 57164347); dog (accession ABY86619.1; GI 166244598); and rabbit (accession AAC23839.1; GI 3242896).

As indicated above, the terms “IL-10”, “IL-10 polypeptide(s), “IL-10 molecule(s)”, “IL-10 agent(s)” and the like are intended to be broadly construed and include, for example, human and non-human IL-10—related polypeptides, including homologs, variants (including muteins), and fragments thereof, as well as IL-10 polypeptides having, for example, a leader sequence (e.g., the signal peptide), and modified versions of the foregoing. In further particular embodiments, IL-10, IL-10 polypeptide(s), and IL-10 agent(s) are agonists.

The IL-10 receptor, a type II cytokine receptor, consists of alpha and beta subunits, which are also referred to as R1 and R2, respectively. Receptor activation requires binding to both alpha and beta. One homodimer of an IL-10 polypeptide binds to alpha and the other homodimer of the same IL-10 polypeptide binds to beta.

The utility of recombinant human IL-10 is frequently limited by its relatively short serum half-life, which can be due to, for example, renal clearance, proteolytic degradation and monomerization in the blood stream. As a result, various approaches have been explored to improve the pharmacokinetic profile of IL-10 without disrupting its dimeric structure and thus adversely affecting its activity. Pegylation of IL-10 results in improvement of certain pharmacokinetic parameters (e.g., serum half-life) and/or enhancement of activity.

As used herein, the terms “pegylated IL-10” and “PEG-IL-10” refer to an IL-10 molecule having one or more polyethylene glycol molecules covalently attached to at least one amino acid residue of the IL-10 protein, generally via a linker, such that the attachment is stable. The terms “monopegylated IL-10” and “mono-PEG-IL-10” indicate that one polyethylene glycol molecule is covalently attached to a single amino acid residue on one subunit of the IL-10 dimer, generally via a linker. As used herein, the terms “dipegylated IL-10” and “di-PEG-IL-10” indicate that at least one polyethylene glycol molecule is attached to a single residue on each subunit of the IL-10 dimer, generally via a linker.

In certain embodiments, the PEG-IL-10 used in the present disclosure is a mono-PEG-IL-10 in which one to nine PEG molecules are covalently attached via a linker to the alpha amino group of the amino acid residue at the N-terminus of one subunit of the IL-10 dimer. Monopegylation on one IL-10 subunit generally results in a non-homogeneous mixture of non-pegylated, monopegylated and dipegylated IL-10 due to subunit shuffling. Moreover, allowing a pegylation reaction to proceed to completion will generally result in non-specific and multi-pegylated IL-10, thus reducing its bioactivity. Thus, particular embodiments of the present disclosure comprise the administration of a mixture of mono- and di-pegylated IL-10 produced by the methods described herein.

In some embodiments, an N-terminal pegylation chemistry strategy can be used that results in pegylation of the N-terminus with approximately 99% specificity over a defined time period (e.g., less than 18 hours). Allowing the chemical reaction to continue beyond that time period results in an increase in lysine side chain pegylation. Several pegylation approaches are described in the Experimental section.

In particular embodiments, the average molecular weight of the PEG moiety is between about 5 kDa and about 50 kDa. Although the method or site of PEG attachment to IL-10 is not critical, in certain embodiments the pegylation does not alter, or only minimally alters, the activity of the IL-10 agent. In certain embodiments, the increase in half-life is greater than any decrease in biological activity. The biological activity of PEG-IL-10 is typically measured by assessing the levels of inflammatory cytokines (e.g., TNF-α or IFN-γ) in the serum of subjects challenged with a bacterial antigen (lipopolysaccharide (LPS)) and treated with PEG-IL-10, as described in U.S. Pat. No. 7,052,686.

IL-10 variants (unmodified by, e.g., pegylation) can be prepared with various objectives in mind, including increasing serum half-life, reducing an immune response against the IL-10, facilitating purification or preparation, decreasing conversion of IL-10 into its monomeric subunits, improving therapeutic efficacy, and lessening the severity or occurrence of side effects during therapeutic use. The amino acid sequence variants are usually predetermined variants not found in nature, although some can be post-translational variants, e.g., glycosylated variants. Any variant of IL-10 can be used provided it retains a suitable level of IL-10 activity. As with wild-type IL-10, these IL-10 variants can be modified (by, e.g., pegylation or Fc fusion) as described herein.

The phrase “conservative amino acid substitution” refers to substitutions that preserve the activity of the protein by replacing an amino acid(s) in the protein with an amino acid with a side chain of similar acidity, basicity, charge, polarity, or size of the side chain. Conservative amino acid substitutions generally entail substitution of amino acid residues within the following groups: 1) L, I, M, V, F; 2) R, K; 3) F, Y, H, W, R; 4) G, A, T, S; 5) Q, N; and 6) D, E. Guidance for substitutions, insertions, or deletions can be based on alignments of amino acid sequences of different variant proteins or proteins from different species. Thus, in addition to any naturally-occurring IL-10 polypeptide, the present disclosure contemplates having 1, 2, 3, 4, 5, 6, 7, 8, 9, or 10 usually no more than 20, 10, or 5 amino acid substitutions, where the substitution is usually a conservative amino acid substitution.

The present disclosure also contemplates active fragments (e.g., subsequences) of mature IL-10 containing contiguous amino acid residues derived from the mature IL-10. The length of contiguous amino acid residues of a peptide or a polypeptide subsequence varies depending on the specific naturally-occurring amino acid sequence from which the subsequence is derived. In general, peptides and polypeptides can be from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length peptide or polypeptide.

Additionally, IL-10 polypeptides can have a defined sequence identity compared to a reference sequence over a defined length of contiguous amino acids (e.g., a “comparison window”). Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Madison, Wis.), or by manual alignment and visual inspection (see, e.g., Current Protocols in Molecular Biology (Ausubel et al., eds. 1995 supplement)).

As an example, a suitable IL-10 polypeptide can comprise an amino acid sequence having at least about 75%, at least about 80%, at least about 85%, at least about 90%, at least about 95%, at least about 98%, or at least about 99%, amino acid sequence identity to a contiguous stretch of from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length peptide or polypeptide.

As discussed further below, the IL-10 polypeptides can be isolated from a non-natural source (e.g., an environment other than its naturally-occurring environment) and can also be recombinantly made (e.g., in a genetically modified host cell such as bacteria, yeast, Pichia, insect cells, and the like), where the genetically modified host cell is modified with a nucleic acid comprising a nucleotide sequence encoding the polypeptide. The IL-10 polypeptides can also be synthetically produced (e.g., by cell-free chemical synthesis).

Nucleic acid molecules encoding the IL-10 agents are contemplated by the present disclosure, including their naturally-occurring and non-naturally occurring isoforms, allelic variants and splice variants. The present disclosure also encompasses nucleic acid sequences that vary in one or more bases from a naturally-occurring DNA sequence but still translate into an amino acid sequence that corresponds to an IL-10 polypeptide due to degeneracy of the genetic code.

The present disclosure also contemplates the use of gene therapy in conjunction with the teachings herein. Gene therapy is effected by delivering genetic material, usually packaged in a vector, to endogenous cells within a subject in order to introduce novel genes, to introduce additional copies of pre-existing genes, to impair the functioning of existing genes, or to repair existing but non-functioning genes. Once inside cells, the nucleic acid is expressed by the cell machinery, resulting in the production of the protein of interest. In the context of the present disclosure, gene therapy is used as a therapeutic to deliver nucleic acid that encodes an IL-10 agent for use in the treatment or prevention of a disease, disorder or condition described herein.

As alluded to above, for gene therapy uses and methods, a cell in a subject can be transformed with a nucleic acid that encodes an IL-10—related polypeptide as set forth herein in vivo. Alternatively, a cell can be transformed in vitro with a transgene or polynucleotide, and then transplanted into a tissue of a subject in order to effect treatment. In addition, a primary cell isolate or an established cell line can be transformed with a transgene or polynucleotide that encodes an IL-10 —related polypeptide, and then optionally transplanted into a tissue of a subject.

### Methods of Production of IL-10

A polypeptide of the present disclosure can be produced by any suitable method, including non-recombinant (e.g., chemical synthesis) and recombinant methods.

A. Chemical Synthesis

Where a polypeptide is chemically synthesized, the synthesis may proceed via liquid-phase or solid-phase. Solid-phase peptide synthesis (SPPS) allows the incorporation of unnatural amino acids and/or peptide/protein backbone modification. Various forms of SPPS, such as 9-fluorenylmethoxycarbonyl (Fmoc) and t-butyloxycarbonyl (Boc), are available for synthesizing polypeptides of the present disclosure. Details of the chemical syntheses are known in the art (e.g., Ganesan A. (2006) Mini Rev. Med. Chem. 6:3-10; and Camarero J. A. et al., (2005) Protein Pept Lett. 12:723-8).

Solid phase peptide synthesis may be performed as described hereafter. The alpha functions (Na) and any reactive side chains are protected with acid-labile or base-labile groups. The protective groups are stable under the conditions for linking amide bonds but can readily be cleaved without impairing the peptide chain that has formed. Suitable protective groups for the a-amino function include, but are not limited to, the following: Boc, benzyloxycarbonyl (Z), O-chlorbenzyloxycarbonyl, bi-phenylisopropyloxycarbonyl, tert-amyloxycarbonyl (Amoc), a, a-dimethyl-3,5-dimethoxy-benzyloxycarbonyl, o-nitrosulfenyl, 2-cyano-t-butoxy-carbonyl, Fmoc, 1-(4,4-dimethyl-2,6-dioxocylohex-1-ylidene)ethyl (Dde) and the like.

Suitable side chain protective groups include, but are not limited to: acetyl, allyl (All), allyloxycarbonyl (Alloc), benzyl (Bzl), benzyloxycarbonyl (Z), t-butyloxycarbonyl (Boc), benzyloxymethyl (Bom), o-bromobenzyloxycarbonyl, t-butyl (tBu), t-butyldimethylsilyl, 2-chlorobenzyl, 2-chlorobenzyloxycarbonyl, 2,6-dichlorobenzyl, cyclohexyl, cyclopentyl, dimethyl-2,6-dioxocyclohex-1-ylidene)ethyl (Dde), isopropyl, 4-methoxy-2,3-6-trimethylbenzylsulfonyl (Mtr), 2,3,5,7,8-pentamethylchroman-6-sulfonyl (Pmc), pivalyl, tetrahydropyran-2-yl, tosyl (Tos), 2,4,6-trimethoxybenzyl, trimethylsilyl and trityl (Trt).

In the solid phase synthesis, the C-terminal amino acid is coupled to a suitable support material. Suitable support materials are those which are inert towards the reagents and reaction conditions for the step-wise condensation and cleavage reactions of the synthesis process and which do not dissolve in the reaction media being used. Examples of commercially-available support materials include styrene/divinylbenzene copolymers which have been modified with reactive groups and/or polyethylene glycol; chloromethylated styrene/divinylbenzene copolymers; hydroxymethylated or aminomethylated styrene/divinylbenzene copolymers; and the like. When preparation of the peptidic acid is desired, polystyrene (1%)-divinylbenzene or TentaGel® derivatized with 4-benzyloxybenzyl-alcohol (Wang-anchor) or 2-chlorotrityl chloride can be used. In the case of the peptide amide, polystyrene (1%) divinylbenzene or TentaGel® derivatized with 5-(4′-aminomethyl)-3′,5′-dimethoxyphenoxy)valeric acid (PAL-anchor) or p-(2,4-dimethoxyphenyl-amino methyl)-phenoxy group (Rink amide anchor) can be used.

The linkage to the polymeric support can be achieved by reacting the C-terminal Fmoc-protected amino acid with the support material by the addition of an activation reagent in ethanol, acetonitrile, N,N-dimethylformamide (DMF), dichloromethane, tetrahydrofuran, N-methylpyrrolidone or similar solvents at room temperature or elevated temperatures (e.g., between 40° C. and 60° C.) and with reaction times of, e.g., 2 to 72 hours.

The coupling of the Na-protected amino acid (e.g., the Fmoc amino acid) to the PAL, Wang or Rink anchor can, for example, be carried out with the aid of coupling reagents such as N,N′-di cyclohexylcarbodiimide (DCC), N,N′-diisopropylcarbodiimide (DIC) or other carbodiimides, 2-(1H-benzotriazol-1-yl)-1,1,3,3 -tetramethyluronium tetrafluoroborate (TBTU) or other uronium salts, O-acyl-ureas, benzotriazol-1-yl-tris-pyrrolidino-phosphonium hexafluorophosphate (PyBOP) or other phosphonium salts, N-hydroxysuccinimides, other N-hydroxyimides or oximes in the presence or absence of 1-hydroxybenzotriazole or 1-hydroxy-7-azabenzotriazole, e.g., with the aid of TBTU with addition of HOBt, with or without the addition of a base such as, for example, diisopropylethylamine (DIEA), triethylamine or N-methylmorpholine, e.g., diisopropylethylamine with reaction times of 2 to 72 hours (e.g., 3 hours in a 1.5 to 3-fold excess of the amino acid and the coupling reagents, for example, in a 2-fold excess and at temperatures between about 10° C. and 50° C., for example, 25° C. in a solvent such as dimethylformamide, N-methylpyrrolidone or dichloromethane, e.g., dimethylformamide).

Instead of the coupling reagents, it is also possible to use the active esters (e.g., pentafluorophenyl, p-nitrophenyl or the like), the symmetric anhydride of the Na-Fmoc-amino acid, its acid chloride or acid fluoride, under the conditions described above.

The Nα-protected amino acid (e.g., the Fmoc amino acid) can be coupled to the 2-chlorotrityl resin in dichloromethane with the addition of DIEA and having reaction times of 10 to 120 minutes, e.g., 20 minutes, but is not limited to the use of this solvent and this base.

The successive coupling of the protected amino acids can be carried out according to conventional methods in peptide synthesis, typically in an automated peptide synthesizer. After cleavage of the Nα-Fmoc protective group of the coupled amino acid on the solid phase by treatment with, e.g., piperidine (10% to 50%) in dimethylformamide for 5 to 20 minutes, e.g., 2×2 minutes with 50% piperidine in DMF and 1×15 minutes with 20% piperidine in DMF, the next protected amino acid in a 3 to 10-fold excess, e.g., in a 10-fold excess, is coupled to the previous amino acid in an inert, non-aqueous, polar solvent such as dichloromethane, DMF or mixtures of the two and at temperatures between about 10° C. and 50° C., e.g., at 25° C. The previously mentioned reagents for coupling the first Nα-Fmoc amino acid to the PAL, Wang or Rink anchor are suitable as coupling reagents. Active esters of the protected amino acid, or chlorides or fluorides or symmetric anhydrides thereof can also be used as an alternative.

At the end of the solid phase synthesis, the peptide is cleaved from the support material while simultaneously cleaving the side chain protecting groups. Cleavage can be carried out with trifluoroacetic acid or other strongly acidic media with addition of 5%-20% V/V of scavengers such as dimethylsulfide, ethylmethylsulfide, thioanisole, thiocresol, m-cresol, anisole ethanedithiol, phenol or water, e.g., 15% v/v dimethylsulfide/ethanedithiol/m-cresol 1:1:1, within 0.5 to 3 hours, e.g., 2 hours. Peptides with fully protected side chains are obtained by cleaving the 2-chlorotrityl anchor with glacial acetic acid/trifluoroethanol/dichloromethane 2:2:6. The protected peptide can be purified by chromatography on silica gel. If the peptide is linked to the solid phase via the Wang anchor and if it is intended to obtain a peptide with a C-terminal alkylamidation, the cleavage can be carried out by aminolysis with an alkylamine or fluoroalkylamine. The aminolysis is carried out at temperatures between about −10° C. and 50° C. (e.g., about 25° C.), and reaction times between about 12 and 24 hours (e.g., about 18 hours). In addition, the peptide can be cleaved from the support by re-esterification, e.g., with methanol.

The acidic solution that is obtained may be admixed with a 3 to 20-fold amount of cold ether or n-hexane, e.g., a 10-fold excess of diethyl ether, in order to precipitate the peptide and hence to separate the scavengers and cleaved protective groups that remain in the ether. A further purification can be carried out by re-precipitating the peptide several times from glacial acetic acid. The precipitate that is obtained can be taken up in water or tert-butanol or mixtures of the two solvents, e.g., a 1:1 mixture of tert-butanol/water, and freeze-dried.

The peptide obtained can be purified by various chromatographic methods, including ion exchange over a weakly basic resin in the acetate form; hydrophobic adsorption chromatography on non-derivatized polystyrene/divinylbenzene copolymers (e.g., Amberlite® XAD); adsorption chromatography on silica gel; ion exchange chromatography, e.g., on carboxymethyl cellulose; distribution chromatography, e.g., on Sephadex® G-25; countercurrent distribution chromatography; or high pressure liquid chromatography (HPLC) e.g., reversed-phase HPLC on octyl or octadecylsilylsilica (ODS) phases.

B. Recombinant Production

Methods describing the preparation of human and mouse IL-10 can be found in, for example, U.S. Patent No. 5,231,012, which teaches methods for the production of proteins having IL-10 activity, including recombinant and other synthetic techniques. IL-10 can be of viral origin, and the cloning and expression of a viral IL-10 from Epstein Barr virus (BCRF1 protein) is disclosed in Moore et al., (1990) Science 248:1230. IL-10 can be obtained in a number of ways using standard techniques known in the art, such as those described herein. Recombinant human IL-10 is also commercially available, e.g., from PeproTech, Inc., Rocky Hill, N.J.

Where a polypeptide is produced using recombinant techniques, the polypeptide may be produced as an intracellular protein or as a secreted protein, using any suitable construct and any suitable host cell, which can be a prokaryotic or eukaryotic cell, such as a bacterial (e.g., E. coli) or a yeast host cell, respectively. Other examples of eukaryotic cells that may be used as host cells include insect cells, mammalian cells, and/or plant cells. Where mammalian host cells are used, they may include human cells (e.g., HeLa, 293, H9 and Jurkat cells); mouse cells (e.g., NIH3T3, L cells, and C127 cells); primate cells (e.g., Cos 1, Cos 7 and CV1); and hamster cells (e.g., Chinese hamster ovary (CHO) cells).

A variety of host-vector systems suitable for the expression of a polypeptide may be employed according to standard procedures known in the art. See, e.g., Sambrook et al., 1989 Current Protocols in Molecular Biology Cold Spring Harbor Press, New York; and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are commercially available.

Vectors can provide for extrachromosomal maintenance in a host cell or can provide for integration into the host cell genome. The expression vector provides transcriptional and translational regulatory sequences, and may provide for inducible or constitutive expression where the coding region is operably-linked under the transcriptional control of the transcriptional initiation region, and a transcriptional and translational termination region. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7).

Expression constructs generally have convenient restriction sites located near the promoter sequence to provide for the insertion of nucleic acid sequences encoding proteins of interest. A selectable marker operative in the expression host may be present to facilitate selection of cells containing the vector. Moreover, the expression construct may include additional elements. For example, the expression vector may have one or two replication systems, thus allowing it to be maintained in organisms, for example, in mammalian or insect cells for expression and in a prokaryotic host for cloning and amplification. In addition, the expression construct may contain a selectable marker gene to allow the selection of transformed host cells. Selectable genes are well known in the art and will vary with the host cell used.

Isolation and purification of a protein can be accomplished according to methods known in the art. For example, a protein can be isolated from a lysate of cells genetically modified to express the protein constitutively and/or upon induction, or from a synthetic reaction mixture by immunoaffinity purification, which generally involves contacting the sample with an anti- protein antibody, washing to remove non-specifically bound material, and eluting the specifically bound protein. The isolated protein can be further purified by dialysis and other methods normally employed in protein purification. In one embodiment, the protein may be isolated using metal chelate chromatography methods. Proteins may contain modifications to facilitate isolation.

The polypeptides may be prepared in substantially pure or isolated form (e.g., free from other polypeptides). The polypeptides can be present in a composition that is enriched for the polypeptide relative to other components that may be present (e.g., other polypeptides or other host cell components). For example, purified polypeptide may be provided such that the polypeptide is present in a composition that is substantially free of other expressed proteins, e.g., less than about 90%, less than about 60%, less than about 50%, less than about 40%, less than about 30%, less than about 20%, less than about 10%, less than about 5%, or less than about 1%.

An IL-10 polypeptide may be generated using recombinant techniques to manipulate different IL-10 —related nucleic acids known in the art to provide constructs capable of encoding the IL-10 polypeptide. It will be appreciated that, when provided a particular amino acid sequence, the ordinary skilled artisan will recognize a variety of different nucleic acid molecules encoding such amino acid sequence in view of her background and experience in, for example, molecular biology.

### Amide Bond Substitutions

In some cases, IL-10 includes one or more linkages other than peptide bonds, e.g., at least two adjacent amino acids are joined via a linkage other than an amide bond. For example, in order to reduce or eliminate undesired proteolysis or other means of degradation, and/or to increase serum stability, and/or to restrict or increase conformational flexibility, one or more amide bonds within the backbone of IL-10 can be substituted.

In another example, one or more amide linkages (—CO—NH—) in IL-10 can be replaced with a linkage which is an isostere of an amide linkage, such as —CH₂NH—, —CH₂S—, —CH₂CH₂—, —CH═CH— (cis and trans), —COCH₂—, —CH(OH)CH₂— or —CH₂SO—. One or more amide linkages in IL-10 can also be replaced by, for example, a reduced isostere pseudopeptide bond. See Couder et al. (1993) Int. J. Peptide Protein Res. 41:181-184. Such replacements and how to effect them are known to those of ordinary skill in the art.

### Amino Acid Substitutions

One or more amino acid substitutions can be made in an IL-10 polypeptide. The following are non-limiting examples:

a) substitution of alkyl-substituted hydrophobic amino acids, including alanine, leucine, isoleucine, valine, norleucine, (S)-2-aminobutyric acid, (S)-cyclohexylalanine or other simple alpha-amino acids substituted by an aliphatic side chain from C₁-C₁₀ carbons including branched, cyclic and straight chain alkyl, alkenyl or alkynyl substitutions;

b) substitution of aromatic-substituted hydrophobic amino acids, including phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkoxy (from C₁-C₄)-substituted forms of the above-listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine;

c) substitution of amino acids containing basic side chains, including arginine, lysine, histidine, ornithine, 2,3-diaminopropionic acid, homoarginine, including alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma'-diethyl-homoarginine. Included also are compounds such as alpha-methyl-arginine, alpha-methyl-2,3-diaminopropionic acid, alpha-methyl-histidine, alpha-methyl-ornithine where the alkyl group occupies the pro-R position of the alpha-carbon. Also included are the amides formed from alkyl, aromatic, heteroaromatic (where the heteroaromatic group has one or more nitrogens, oxygens or sulfur atoms singly or in combination), carboxylic acids or any of the many well-known activated derivatives such as acid chlorides, active esters, active azolides and related derivatives, and lysine, ornithine, or 2,3-diaminopropionic acid;

d) substitution of acidic amino acids, including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids;

e) substitution of side chain amide residues, including asparagine, glutamine, and alkyl or aromatic substituted derivatives of asparagine or glutamine; and

f) substitution of hydroxyl-containing amino acids, including serine, threonine, homoserine, 2,3-diaminopropionic acid, and alkyl or aromatic substituted derivatives of serine or threonine.

In some cases, IL-10 comprises one or more naturally occurring non-genetically encoded L-amino acids, synthetic L-amino acids, or D-enantiomers of an amino acid. For example, IL-10 can comprise only D-amino acids. For example, an IL-10 polypeptide can comprise one or more of the following residues: hydroxyproline, β-alanine, o-aminobenzoic acid, m-aminobenzoic acid, p-aminobenzoic acid, m-aminomethylbenzoic acid, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylalanine 3-benzothienyl alanine, 4-chlorophenylalanine, 2-fluorophenylalanine, 3-fluorophenylalanine, 4-fluorophenylalanine, penicillamine, 1,2,3,4-tetrahydroisoquinoline-3-carboxylic acid, β-2-thienylalanine, methionine sulfoxide, homoarginine, N-acetyl lysine, 2,4-diamino butyric acid, rho-aminophenylalanine, N-methylvaline, homocysteine, homoserine, ε-amino hexanoic acid, ω-aminohexanoic acid, ω-aminoheptanoic acid, ω-aminooctanoic acid, ω-aminodecanoic acid, ω-aminotetradecanoic acid, cyclohexylalanine, α,γ-diaminobutyric acid, α,β-diaminopropionic acid, δ-amino valeric acid, and 2,3-diaminobutyric acid.

### Additional Modifications

A cysteine residue or a cysteine analog can be introduced into an IL-10 polypeptide to provide for linkage to another peptide via a disulfide linkage or to provide for cyclization of the IL-10 polypeptide. Methods of introducing a cysteine or cysteine analog are known in the art; see, e.g., U.S. Pat. No. 8,067,532.

An IL-10 polypeptide can be cyclized. One or more cysteines or cysteine analogs can be introduced into an IL-10 polypeptide, where the introduced cysteine or cysteine analog can form a disulfide bond with a second introduced cysteine or cysteine analog. Other means of cyclization include introduction of an oxime linker or a lanthionine linker; see, e.g., U.S. Pat. No. 8,044,175. Any combination of amino acids (or non-amino acid moieties) that can form a cyclizing bond can be used and/or introduced. A cyclizing bond can be generated with any combination of amino acids (or with an amino acid and —(CH2)ₙ-CO— or —(CH2)ₙ-C₆H₄—CO—) with functional groups which allow for the introduction of a bridge. Some examples are disulfides, disulfide mimetics such as the —(CH2)n- carba bridge, thioacetal, thioether bridges (cystathionine or lanthionine) and bridges containing esters and ethers. In these examples, n can be any integer, but is frequently less than ten.

Other modifications include, for example, an N-alkyl (or aryl) substitution (ψ[CONR]), or backbone crosslinking to construct lactams and other cyclic structures. Other derivatives include C-terminal hydroxymethyl derivatives, o-modified derivatives (e.g., C-terminal hydroxymethyl benzyl ether), N-terminally modified derivatives including substituted amides such as alkylamides and hydrazides.

In some cases, one or more L-amino acids in an IL-10 polypeptide is replaced with one or more D-amino acids.

In some cases, an IL-10 polypeptide is a retroinverso analog (see, e.g., Sela and Zisman (1997) FASEB J. 11:449). Retro-inverso peptide analogs are isomers of linear polypeptides in which the direction of the amino acid sequence is reversed (retro) and the chirality, D- or L-, of one or more amino acids therein is inverted (inverso), e.g., using D-amino acids rather than L-amino acids.)See, e.g., Jameson et al. (1994) Nature 368:744; and Brady et al. (1994) Nature 368:692).

An IL-10 polypeptide can include a “Protein Transduction Domain” (PTD), which refers to a polypeptide, polynucleotide, carbohydrate, or organic or inorganic molecule that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of an IL-10 polypeptide, while in other embodiments, a PTD is covalently linked to the carboxyl terminus of an IL-10 polypeptide. Exemplary protein transduction domains include, but are not limited to, a minimal undecapeptide protein transduction domain (corresponding to residues 47-57 of HIV-1 TAT comprising YGRKKRRQRRR; SEQ ID NO:1); a polyarginine sequence comprising a number of arginine residues sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:2); Transportan GWTLNSAGYLLGKINLKALAALAKKIL (SEQ ID NO:3); KALAWEAKLAKALAKALAKHLAKALAKALKCEA (SEQ ID NO:4); and RQIKIWFQNRRMKWKK (SEQ ID NO:5). Exemplary PTDs include, but are not limited to, YGRKKRRQRRR (SEQ ID NO:1), RKKRRQRRR (SEQ ID NO:6); an arginine homopolymer of from 3 arginine residues to 50 arginine residues; exemplary PTD domain amino acid sequences include, but are not limited to, any of the following: YGRKKRRQRRR (SEQ ID NO:1); RKKRRQRR (SEQ ID NO:7); YARAAARQARA (SEQ ID NO:8); THRLPRRRRRR (SEQ ID NO:9); and GGRRARRRRRR (SEQ ID NO:10).

The carboxyl group COR3 of the amino acid at the C-terminal end of an IL-10 polypeptide can be present in a free form (R3=OH) or in the form of a physiologically-tolerated alkaline or alkaline earth salt such as, e.g., a sodium, potassium or calcium salt. The carboxyl group can also be esterified with primary, secondary or tertiary alcohols such as, e.g., methanol, branched or unbranched C1-C6-alkyl alcohols, e.g., ethyl alcohol or tert-butanol. The carboxyl group can also be amidated with primary or secondary amines such as ammonia, branched or unbranched C1-C6-alkylamines or C1-C6 di-alkylamines, e.g., methylamine or dimethylamine.

The amino group of the amino acid NR1R2 at the N-terminus of an IL-10 polypeptide can be present in a free form (R1=H and R2=H) or in the form of a physiologically-tolerated salt such as, e.g., a chloride or acetate. The amino group can also be acetylated with acids such that R1=H and R2=acetyl, trifluoroacetyl, or adamantyl. The amino group can be present in a form protected by amino-protecting groups conventionally used in peptide chemistry, such as those provided above (e.g., Fmoc, Benzyloxy-carbonyl (Z), Boc, and Alloc). The amino group can be N-alkylated in which R₁ and/or R₂═C₁-C₆ alkyl or C₂-C₈ alkenyl or C₇-C₉ aralkyl. Alkyl residues can be straight-chained, branched or cyclic (e.g., ethyl, isopropyl and cyclohexyl, respectively).

### Considerations for the Production of IL-10

If IL-10 is produced in inclusion bodies in a bacterial (e.g., E. coli) expression system, it must be denatured, refolded, and purified from contaminants. Such contaminants include host proteins, modified variants of IL-10 (e.g., IL-10 monomers acetylated at one or more lysine residues), heterodimers of such variants (e.g., acetylated IL-10 monomers bound to non-acetylated IL-10 monomers), and covalently bonded IL-10 homodimers. Thus, IL-10 must be purified to obtain essentially pure non-covalently bonded dimeric IL-10 free of the acetylated homodimer, heterodimer variants and covalent dimers. U.S. Pat. No. 5,710,251 describes purification processes that may be employed after IL-10 produced in inclusion bodies in a bacterial expression system is denatured and refolded.

In order to be successful, a purification process must, in part, result in the recovery of biologically active and/or soluble protein in high yield. This is accomplished by optimizing the solubilization and/or refolding processes with which the protein in the inclusion bodies is subjected. Refolding of proteins from inclusion bodies is affected by several factors, including solubilization of inclusion bodies by denaturants, removal of the denaturant, and assistance of refolding by certain small molecule additives. Various methodologies associated with the solubilization and refolding processes can be found in, for example, Rudolph R. and Lilie, H. (1996) FASEB 10:49-56; Lilie, H., et al. (1998) Current Opinion Biotechnol. 9:497-501; Middelberg, A. (2002) Trends Biotechnol. 20(10):437-443; Hevehan, D. L. and Clark, E. D. B. (1997) Biotechnol. Bioeng. 54(3):221-30; De Bernardez Clark, E. (1998) Current Opinion Biotechnol. 9:157-63; Tsumoto, K. et al. (2003) Protein Expression & Purification 28:1-8.

The solubilization and refolding processes may be carried out in three phases:

1) Isolation of Inclusion Bodies. Inclusion bodies have a relatively high density and, therefore, can be pelleted by centrifugation. Cells are usually disrupted by high pressure homogenization (optionally following a lysozyme treatment). Cell lysis must be complete in order to prevent intact cells containing inclusion bodies from accumulating together in the form of a sediment. Subsequent to centrifugation, in order to remove contaminants from the pellet it may be washed with buffer containing either low concentrations of chaotropic agents (e.g., 0.5-1 M guanidine-HCl or urea) or detergents (e.g., 1% Triton X-100 or 1 mg/mL sodium deoxycholate).

2) Solubilization of Aggregated Proteins. Solubilization must result in monomolecular dispersion and minimum non-native intra- or inter-chain interactions. Choice of solubilizing agents, e.g., urea, guanidine HC1, or detergents, plays a key role in solubilization efficiency, in the structure of the proteins in denatured state, and in subsequent refolding.

In one methodology, the above-described washed inclusion bodies may be resuspended and incubated in buffer containing a strong denaturant and a reducing agent (e.g., 20 mM DTT or b-mercaptoethanol). The reducing agent keeps all cysteines in the reduced state and cleaves disulfide bonds formed during the preparation. Incubation temperatures above 30° C. are typically used to facilitate the solubilization process. Optimal conditions for solubilization are protein-specific and thus must be determined for each protein by, for example, conducting small-scale experiments (1-2 mL) to screen for different variables. Particular variables for solubilization, along with potential starting values (listed in parentheses), include the following: a) buffer composition, such as pH and ionic strength (50 mM Tris-HCl, pH 7.5); b) incubation temperature (30° C.); c) incubation time (60 mins); d) concentration of solubilizing agent (6 M guanidine-HCl or 8 M urea; e) total protein concentration (1-2 mg/mL); and f) ratio of solubilizing agent to protein of interest.

Subsequent to solubilization, the solution may be centrifuged (e.g., 30 min at >100,000 g) to remove remaining aggregates which could act as nuclei to trigger aggregation during refolding. Typically, ultracentrifugation provides the best results.

3) Refolding of Solubilized Proteins. Protein refolding is not a single reaction and competes with other reactions, such as misfolding and aggregation, leading to inactive proteins. The rate of refolding and other reactions is determined by both the procedure used to reduce denaturant concentration and the solvent condition. Several protein refolding kits and related technologies are commercially available (e.g., Pierce Protein Refolding Kit (Thermo Fisher Scientific; Rockford, Ill.) and FoldIt® protein folding screen (Hampton Research Inc.; Aliso Viejo, Calif.)) and are known to the skilled artisan.

Refolding of solubilized proteins is initiated by the removal of the denaturant. The efficiency of refolding depends on the competition between correct folding and aggregation. In order to slow down the aggregation process, refolding is usually carried out at low protein concentrations (e.g., 10-100 mg/mL). The conditions used for refolding, including buffer composition (e.g., pH and ionic strength), temperature, and additive components, must be optimized for each individual protein. Certain small molecule additives are effective in facilitating folding and stabilizing proteins or increasing solubility both in vitro and in vivo. Thus, small molecules additives, sometimes referred to as chemical chaperones, can increase the recovery of active proteins and the efficiency of protein folding.

If proteins contain disulfide bonds, the refolding buffer has to be supplemented with a redox system. By way of example, the addition of a mixture of reduced and oxidized forms (1-3 mM reduced thiol and a 5:1 to 1:1 ratio of reduced to oxidized thiol) of low molecular weight thiol reagent generally provides the appropriate redox potential to allow formation and reshuffling of disulfide bonds. The most commonly used redox shuffling reagents are reduced and oxidized glutathione; others include cysteine and cysteamine. Alternatively, proteins can be completely oxidized in the presence of a large excess of oxidized glutathione, followed by dilution in refolding buffer containing catalytic amounts of reduced glutathione.

The skilled artisan is familiar with different methods for the refolding of proteins, including the following:

(a) Dialysis: During dialysis, the most commonly used method for the removal of the solubilizing agent, the concentration of the solubilizing agent decreases slowly, which allows the protein to refold optimally. The ratio of the volumes of the sample and the dialysis buffer should be as such that at the equilibrium concentration of the solubilizing agent the protein has completely refolded.

(b) Slow Dilution: With this process, the concentration of the solubilizing agent is decreased by dilution, allowing the protein to refold. This dilution process is usually carried out slowly by step-wise addition of buffer or by continuous addition using a pump.

(c) Rapid Dilution: In general, during the dialysis and slow dilution processes, the protein is exposed for an extended period of time to an intermediate concentration of the solubilizing agent (e.g., 2-4 M urea or guanidine-HCl) where it is not yet folded but no longer denatured and thus is extremely prone to aggregation. This propensity for aggregation often can be prevented by the rapid dilution of the solubilized protein solution into the refolding buffer. Aggregation can also be limited by adding mild solubilizing agents to the refolding buffer, such as non-detergent sulfobetaines.

(d) Pulse Renaturation: To maintain a low concentration of the unfolded protein and thus limiting aggregation, aliquots (“pulses”) of denatured protein can be added at defined time points to the refolding buffer. The time intervals between two pulses have to be optimized for each individual protein. The process can be stopped when the concentration of denaturant reaches a critical level with respect to refolding of the specific protein.

(e) Chromatography: Using this method, the solubilizing agent is removed using a chromatographic step. Different chromatography methods may be used, including size exclusion chromatography, ion exchange chromatography, and affinity chromatography. The denaturant is removed while the protein slowly migrates through the column or is bound to the matrix. This usually gives a high yield of active protein even at protein concentrations in the mg/mL range. Alternatively, chromatography can be conducted under denaturing conditions before protein refolding.

### Amino Acids

The addition of particular amino acids to the refolding buffer has been observed to have several beneficial effects during the refolding process, including improving the solubility of proteins and inhibiting protein aggregation. Exemplary amino acids include proline, arginine hydrochloride (ArgHCl), arginine (Arg), arginineamide and glycineamide. While the underlying mechanism of action by which these amino acids cause their effects is not entirely clear, an understanding of their mechanism is not required in order to practice the present disclosure. [See Yamaguchi, H. et al., Biomolecules 2014, 4:235-51].

Arginine has been applied for the refolding of a number of proteins from inclusion bodies, including casein kinase II, gamma interferon, p53 tumor suppressor protein, and interleukin-21. Arginine, which is generally considered to be a volume expander, may exert its effects by inhibiting aggregation due to its moderate binding to proteins. Arginineamide and glycineamide have been reported to be moderate chaotropic agents that bind to different sites than arginine, which leads to different inhibitory abilities. In contrast, it has been proposed that proline enables proteins to refold to their native conformation by inhibiting protein aggregation via binding to the folding intermediate(s) and trapping the folding intermediate(s) in the supramolecular assembly with proline (Samuel, D. et al., Protein Sci. 2000, 9:344-52).

As detailed in the Experimental section, despite the fact that arginine is often used in solvents for refolding proteins by dialysis or dilution, there is little discussion in the scientific or patent literature regarding the addition of arginine to a refold buffer for use in the production of IL-10. (see, e.g., Tsumoto, K. et al., (2004) Biotechnol. Prog. 20:1301-08). The data in Example 2 indicate that low concentrations of L-Arginine were found to positively impact IL-10 yield. In particular, the addition of 0.01-0.1 M arginine to a refold buffer containing 0.15 mg/mL unfolded rHuIL-10 led to at least a two-fold increase of properly folded, dimeric IL-10.

Particular Modifications to Enhance and/or Mimic IL-10 Function

It is frequently beneficial, and sometimes imperative, to improve one of more physical properties of the treatment modalities disclosed herein (e.g., IL-10) and/or the manner in which they are administered. Improvements of physical properties include, for example, modulating immunogenicity; methods of increasing water solubility, bioavailability, serum half-life, and/or therapeutic half-life; and/or modulating biological activity. Certain modifications may also be useful to, for example, raise of antibodies for use in detection assays (e.g., epitope tags) and to provide for ease of protein purification. Such improvements must generally be imparted without adversely impacting the bioactivity of the treatment modality and/or increasing its immunogenicity.

Pegylation of IL-10 is one particular modification contemplated by the present disclosure, while other modifications include, but are not limited to, glycosylation (N- and O-linked); polysialylation; albumin fusion molecules comprising serum albumin (e.g., human serum albumin (HSA), cyno serum albumin, or bovine serum albumin (BSA)); albumin binding through, for example a conjugated fatty acid chain (acylation); and Fc-fusion proteins.

Pegylation: The clinical effectiveness of protein therapeutics is often limited by short plasma half-life and susceptibility to protease degradation. Studies of various therapeutic proteins (e.g., filgrastim) have shown that such difficulties may be overcome by various modifications, including conjugating or linking the polypeptide sequence to any of a variety of nonproteinaceous polymers, e.g., polyethylene glycol (PEG), polypropylene glycol, or polyoxyalkylenes. This is frequently effected by a linking moiety covalently bound to both the protein and the nonproteinaceous polymer, e.g., a PEG. Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

In addition to the beneficial effects of pegylation on pharmacokinetic parameters, pegylation itself may enhance activity. For example, PEG-IL-10 has been shown to be more efficacious against certain cancers than unpegylated IL-10 (see, e.g., EP 206636A2). Certain embodiments of the present disclosure contemplate the use of a relatively small PEG (e.g., 5 kDa) that improves the pharmacokinetic profile of the IL-10 molecule without causing untoward adverse effects; such PEG-IL-10 molecules are especially efficacious for chronic use.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range, and examples are set forth elsewhere herein; by way of example, certain embodiments have molecular weights between 5 kDa and 20 kDa, while other embodiments have molecular weights between 4 kDa and 10 kDa.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. Exemplary reaction conditions are described throughout the specification. Cation exchange chromatography may be used to separate conjugates, and a fraction is then identified which contains the conjugate having, for example, the desired number of PEGs attached, purified free from unmodified protein sequences and from conjugates having other numbers of PEGs attached.

Pegylation most frequently occurs at the alpha amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein. PEG may be bound to a polypeptide of the present disclosure via a terminal reactive group (a “spacer”) which mediates a bond between the free amino or carboxyl groups of one or more of the polypeptide sequences and polyethylene glycol. The PEG having the spacer which may be bound to the free amino group includes N-hydroxysuccinylimide polyethylene glycol which may be prepared by activating succinic acid ester of polyethylene glycol with N-hydroxysuccinylimide. Another activated polyethylene glycol which may be bound to a free amino group is 2,4-bis(O-methoxypolyethyleneglycol)-6-chloro-s-triazine, which may be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art may be used to terminate the reaction. In some embodiments the reaction is terminated by acidifying the reaction mixture and freezing at, e.g., −20° C. Pegylation of various molecules is discussed in, for example, U.S. Pat. Nos. 5,252,714; 5,643,575; 5,919,455; 5,932,462; and 5,985,263. PEG-IL-10 is described in, e.g., U.S. Pat. No. 7,052,686. Specific reaction conditions contemplated for use herein are set forth in the Experimental section.

The present disclosure also contemplates the use of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix' XTEN technology; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

Glycosylation: For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present.

Glycosylation can dramatically affect the physical properties (e.g., solubility) of polypeptides such as IL-10 and can also be important in protein stability, secretion, and subcellular localization. Glycosylated polypeptides may also exhibit enhanced stability or may improve one or more pharmacokinetic properties, such as half-life. In addition, solubility improvements can, for example, enable the generation of formulations more suitable for pharmaceutical administration than formulations comprising the non-glycosylated polypeptide.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants.

The polypeptide sequences of the present disclosure may optionally be altered through changes at the nucleic acid level, particularly by mutating the nucleic acid encoding the polypeptide at preselected bases such that codons are generated that will translate into the desired amino acids.

Polysialylation: The present disclosure also contemplates the use of polysialylation, the conjugation of polypeptides to the naturally occurring, biodegradable α-(2→8) linked polysialic acid (“PSA”) in order to improve the polypeptides' stability and in vivo pharmacokinetics.

Albumin Fusion: Additional suitable components and molecules for conjugation include albumins such as human serum albumin (HSA), cyno serum albumin, and bovine serum albumin (BSA).

According to the present disclosure, albumin may be conjugated to a drug molecule (e.g., a polypeptide described herein) at the carboxyl terminus, the amino terminus, both the carboxyl and amino termini, and internally (see, e.g., U.S. Pat. No. 5,876,969 and U.S. Pat. No. 7,056,701).

In the HSA—drug molecule conjugates contemplated by the present disclosure, various forms of albumin may be used, such as albumin secretion pre-sequences and variants thereof, fragments and variants thereof, and HSA variants. Such forms generally possess one or more desired albumin activities. In additional embodiments, the present disclosure involves fusion proteins comprising a polypeptide drug molecule fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. In some embodiments, the indirect fusion is effected by a linker, such as a peptide linker or modified version thereof.

As alluded to above, fusion of albumin to one or more polypeptides of the present disclosure can, for example, be achieved by genetic manipulation, such that the nucleic acid coding for HSA, or a fragment thereof, is joined to the nucleic acid coding for the one or more polypeptide sequences.

Alternative Albumin Binding Strategies: Several albumin—binding strategies have been developed as alternatives to direct fusion and may be used with the IL-10 agents described herein. By way of example, the present disclosure contemplates albumin binding through a conjugated fatty acid chain (acylation) and fusion proteins which comprise an albumin binding domain (ABD) polypeptide sequence and the sequence of one or more of the polypeptides described herein.

Conjugation with Other Molecules: Additional suitable components and molecules for conjugation include, for example, thyroglobulin; tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Thus, the present disclosure contemplates conjugation of one or more additional components or molecules at the N- and/or C-terminus of a polypeptide sequence, such as another polypeptide (e.g., a polypeptide having an amino acid sequence heterologous to the subject polypeptide), or a carrier molecule. Thus, an exemplary polypeptide sequence can be provided as a conjugate with another component or molecule.

An IL-10 polypeptide may also be conjugated to large, slowly metabolized macromolecules such as proteins; polysaccharides, such as sepharose, agarose, cellulose, or cellulose beads; polymeric amino acids such as polyglutamic acid, or polylysine; amino acid copolymers; inactivated virus particles; inactivated bacterial toxins such as toxoid from diphtheria, tetanus, cholera, or leukotoxin molecules; inactivated bacteria; and dendritic cells. Such conjugated forms, if desired, can be used to produce antibodies against a polypeptide of the present disclosure.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

Fc-fusion Molecules: In certain embodiments, the amino- or carboxyl- terminus of a polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration.

Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

Other Modifications: The present disclosure contemplates the use of other modifications, currently known or developed in the future, of IL-10 to improve one or more properties. One such method involves modification of the polypeptide sequences by hesylation, which utilizes hydroxyethyl starch derivatives linked to other molecules in order to modify the polypeptide sequences' characteristics. Various aspects of hesylation are described in, for example, U.S. Patent Appln. Nos. 2007/0134197 and 2006/0258607.

The present disclosure also contemplates fusion molecules comprising Small Ubiquitin-like Modifier (SUMO) as a fusion tag (LifeSensors, Inc.; Malvern, Pa.). Fusion of a polypeptide described herein to SUMO may convey several beneficial effects, including enhancement of expression, improvement in solubility, and/or assistance in the development of purification methods. SUMO proteases recognize the tertiary structure of SUMO and cleave the fusion protein at the C-terminus of SUMO, thus releasing a polypeptide described herein with the desired N-terminal amino acid.

The present disclosure also contemplates the use of PASylation™ (XL-Protein GmbH (Freising, Germany)). This technology expands the apparent molecular size of a protein of interest, without having a negative impact on the therapeutic bioactivity of the protein, beyond the pore size of the renal glomeruli, thereby decreasing renal clearance of the protein.

Linkers: Any of the foregoing components and molecules used to modify the polypeptide sequences of the present disclosure may optionally be conjugated via a linker. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids.

Examples of flexible linkers include glycine polymers (G), glycine-alanine polymers, alanine-serine polymers, glycine-serine polymers (for example, (GₘSₒ)ₙ, (GSGGS)ₙ (SEQ ID NO:11), (GₘSₒGₘ)ₙ, (GₘSₒGₘSₒGₘ)ₙ (SEQ ID NO:12), (GSGGSₘ)ₙ (SEQ ID NO:13), (GSGSₘG)ₙ (SEQ ID NO:14) and (GGGSₘ)ₙ (SEQ ID NO:15), and combinations thereof, where m, n, and o are each independently selected from an integer of at least 1 to 20, e.g., 1-18, 2-16, 3-14, 4-12, 5-10, 1, 2, 3, 4, 5, 6, 7, 8,9, or 10), and other flexible linkers. Glycine and glycine-serine polymers are relatively unstructured, and therefore may serve as a neutral tether between components. Examples of flexible linkers include, but are not limited to GGSG (SEQ ID NO:16), GGSGG (SEQ ID NO:17), GSGSG (SEQ ID NO:14), GSGGG (SEQ ID NO:18), GGGSG (SEQ ID NO:19), and GSSSG (SEQ ID NO:20).

Additional examples of flexible linkers include glycine polymers (G)ₙ or glycine-serine polymers (e.g., (GS), (GSGGS)ₙ(SEQ ID NO:11), (GGGS)ₙ (SEQ ID NO:21) and (GGGGS)ₙ(SEQ ID NO:22), where n=1 to 50, for example, 1, 2, 3, 4, 5, 6, 7, 8, 9, 10, 10-20, 20-30, 30-50). Exemplary flexible linkers include, but are not limited to GGGS (SEQ ID NO: 21), GGGGS (SEQ ID NO: 22), GGSG (SEQ ID NO: 16), GGSGG (SEQ ID NO: 17), GSGSG (SEQ ID NO: 12), GSGGG (SEQ ID NO: 18), GGGSG (SEQ ID NO: 19), and GSSSG (SEQ ID NO: 20). A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the Polypeptides disclosed herein. As described herein, the heterologous amino acid sequence may be a signal sequence and/or a fusion partner, such as, albumin, Fc sequence, and the like.

### Therapeutic and Prophylactic Uses

The present disclosure contemplates the use of the IL-10 polypeptides described herein (e.g., PEG-IL-10) in the treatment or prevention of a broad range of diseases, disorders and/or conditions, and/or the symptoms thereof. While particular uses are described hereafter, it is to be understood that the present disclosure is not so limited. Furthermore, although general categories of particular diseases, disorders and conditions are set forth hereafter, some of the diseases, disorders and conditions may be a member of more than one category (e.g., cancer- and fibrotic-related disorders), and others may not be a member of any of the disclosed categories.

Fibrotic Disorders and Cancer. In accordance with the present disclosure, an IL-10 molecule can be used to treat or prevent a proliferative condition or disorder, including a cancer, for example, cancer of the uterus, cervix, breast, prostate, testes, gastrointestinal tract (e.g., esophagus, oropharynx, stomach, small or large intestines, colon, or rectum), kidney, renal cell, bladder, bone, bone marrow, skin, head or neck, liver, gall bladder, heart, lung, pancreas, salivary gland, adrenal gland, thyroid, brain (e.g., gliomas), ganglia, central nervous system (CNS) and peripheral nervous system (PNS), and cancers of the hematopoietic system and the immune system (e.g., spleen or thymus). The present disclosure also provides methods of treating or preventing other cancer-related diseases, disorders or conditions, including, for example, immunogenic tumors, non-immunogenic tumors, dormant tumors, virus-induced cancers (e.g., epithelial cell cancers, endothelial cell cancers, squamous cell carcinomas and papillomavirus), adenocarcinomas, lymphomas, carcinomas, melanomas, leukemias, myelomas, sarcomas, teratocarcinomas, chemically-induced cancers, metastasis, and angiogenesis. The disclosure contemplates reducing tolerance to a tumor cell or cancer cell antigen, e.g., by modulating activity of a regulatory T-cell and/or a CD8+ T-cell (see, e.g., Ramirez-Montagut, et al. (2003) Oncogene 22:3180-87; and Sawaya, et al. (2003) New Engl. J. Med. 349:1501-09). In particular embodiments, the tumor or cancer is colon cancer, ovarian cancer, breast cancer, melanoma, lung cancer, glioblastoma, or leukemia. The use of the term(s) cancer-related diseases, disorders and conditions is meant to refer broadly to conditions that are associated, directly or indirectly, with cancer, and includes, e.g., angiogenesis and precancerous conditions such as dysplasia.

In some embodiments, the present disclosure provides methods for treating a proliferative condition, cancer, tumor, or precancerous condition with an IL-10 molecule and at least one additional therapeutic or diagnostic agent, examples of which are set forth elsewhere herein.

Cardiovascular Diseases. In particular embodiments, the present disclosure contemplates the use of the IL-10 polypeptides (e.g., PEG-IL-10) described herein to treat and/or prevent cardiovascular diseases, disorders and conditions, as well as disorders associated therewith, resulting from hypercholesterolemia and aberrant lipid profile.

As used herein, the terms “cardiovascular disease”, “heart disease” and the like refer to any disease that affects the cardiovascular system, primarily cardiac disease, vascular diseases of the brain and kidney, and peripheral arterial diseases. Cardiovascular disease is a constellation of diseases that includes coronary heart disease (e.g., ischemic heart disease or coronary artery disease), atherosclerosis, cardiomyopathy, hypertension, hypertensive heart disease, cor pulmonale, cardiac dysrhythmias, endocarditis, cerebrovascular disease, and peripheral arterial disease. Cardiovascular disease is the leading cause of deaths worldwide, and while it usually affects older adults, the antecedents of cardiovascular disease, notably atherosclerosis, begin in early life.

Particularly contemplated by the present disclosure are embodiments wherein the cardiovascular disease comprises a hyperlipidemia (or hyperlipoproteinemia), conditions characterized by abnormally elevated levels of lipids and/or lipoproteins in the blood. Non-limiting examples of hyperlipidemias include dyslipidemia, hypercholesterolemia (e.g., familial hypercholesterolemia), hyperglyceridemia, hypertriglyceridemia, hyperlipoproteinemia, hyperchylomicronemia, and combined hyperlipidemia. Hyperlipoproteinemias include, for example, hyperlipoproteinemia type Ia, hyperlipoproteinemia type Ib, hyperlipoproteinemia type Ic, hyperlipoproteinemia type IIa, hyperlipoproteinemia type IIb, hyperlipoproteinemia type III, hyperlipoproteinemia type IV, and hyperlipoproteinemia type V.

Thrombosis and Thrombotic Conditions. In other embodiments, the present disclosure contemplates the use of the IL-10 polypeptides (e.g., PEG-IL-10) described herein to treat and/or prevent thrombosis and thrombotic diseases, disorders and conditions, as well as disorders associated therewith, resulting from hypercholesterolemia and aberrant lipid profile.

Thrombosis is generally categorized as venous or arterial, each of which can be presented by several subtypes. Venous thrombosis includes deep vein thrombosis (DVT), portal vein thrombosis, renal vein thrombosis, jugular vein thrombosis, Budd-Chiari syndrome, Paget-Schroetter disease, and cerebral venous sinus thrombosis. Arterial thrombosis includes stroke and myocardial infarction.

Immune and Inflammatory Conditions. As used herein, terms such as “immune disease”, “immune condition”, “immune disorder”, “inflammatory disease”, “inflammatory condition”, “inflammatory disorder” and the like are meant to broadly encompass any immune- or inflammatory-related condition (e.g., pathological inflammation and autoimmune diseases). Such conditions frequently are inextricably intertwined with other diseases, disorders and conditions. By way of example, an “immune condition” may refer to proliferative conditions, such as cancer, tumors, and angiogenesis; including infections (acute and chronic), tumors, and cancers that resist eradication by the immune system.

A non-limiting list of immune- and inflammatory-related diseases, disorders and conditions which may, for example, be caused by inflammatory cytokines, include, arthritis (e.g., rheumatoid arthritis), kidney failure, lupus, asthma, psoriasis, colitis, pancreatitis, allergies, fibrosis, surgical complications (e.g., where inflammatory cytokines prevent healing), anemia, and fibromyalgia. Other diseases and disorders which may be associated with chronic inflammation include congestive heart failure, stroke, aortic valve stenosis, arteriosclerosis, osteoporosis, infections, inflammatory bowel disease (e.g., Crohn's disease and ulcerative colitis), allergic contact dermatitis and other eczemas, systemic sclerosis, transplantation, multiple sclerosis and neurodegenerative disorders (e.g., Alzheimer's disease and Parkinson's disease).

The present disclosure includes embodiments wherein the IL-10 agents described herein (e.g., PEG-IL-10) are used in the treatment and/or prevention of a vasculitis, including, without limitation, Buerger's disease (thromboangiitis obliterans), cerebral vasculitis (central nervous system vasculitis), Churg-Strauss arteritis, cryoglobulinemia, essential cryoglobulinemic vasculitis, giant cell (temporal) arteritis, Henoch-Schonlein purpura, hypersensitivity vasculitis (allergic vasculitis), Kawasaki disease, microscopic polyarteritis/polyangiitis, polyarteritis nodosa, polymyalgia rheumatica (PMR), rheumatoid vasculitis, Takayasu arteritis, thrombophlebitis, Wegener's granulomatosis; and vasculitis secondary to connective tissue disorders like systemic lupus erythematosus, rheumatoid arthritis, relapsing polychondritis, Behcet's disease, or other connective tissue disorders; and vasculitis secondary to viral infection.

Other embodiments are directed to an inflammatory heart disease, including endocarditis, inflammatory cardiomegaly, and myocarditis.

Viral Diseases. The present disclosure contemplates the use of the IL-10 polypeptides in the treatment and/or prevention of any viral disease, disorder or condition for which treatment with IL-10 may be beneficial. Examples of viral diseases, disorders and conditions that are contemplated include hepatitis B, hepatitis C, HIV, herpes virus and cytomegalovirus (CMV).

Treatment of many viral diseases (e.g., HIV) comprise the administration of combinations of agents, including agents that act through different mechanisms of action, and the present disclosure contemplates the use of the IL-10 polypeptides described herein as a component of such combination therapy.

Fibrotic Disorders: The present disclosure also provides methods of treating or preventing fibrotic diseases, disorders and conditions. As used herein, the phrase “fibrotic diseases, disorders and conditions”, and similar terms (e.g., “fibrotic disorders”) and phrases, is to be construed broadly such that it includes any condition which may result in the formation of fibrotic tissue or scar tissue (e.g., fibrosis in one or more tissues). By way of example, injuries (e.g., wounds) that may give rise to scar tissue include wounds to the skin, eye, lung, kidney, liver, central nervous system, and cardiovascular system. The phrase also encompasses scar tissue formation resulting from stroke, and tissue adhesion, for example, as a result of injury or surgery.

As used herein the term “fibrosis” refers to the formation of fibrous tissue as a reparative or reactive process, rather than as a normal constituent of an organ or tissue. Fibrosis is characterized by fibroblast accumulation and collagen deposition in excess of normal deposition in any particular tissue.

Fibrotic disorders include, but are not limited to, fibrosis arising from wound healing, systemic and local scleroderma, atherosclerosis, restenosis, pulmonary inflammation and fibrosis, idiopathic pulmonary fibrosis, interstitial lung disease, liver cirrhosis, fibrosis as a result of chronic hepatitis B or C infection, kidney disease (e.g., glomerulonephritis), heart disease resulting from scar tissue, keloids and hypertrophic scars, and eye diseases such as macular degeneration, and retinal and vitreal retinopathy. Additional fibrotic diseases include chemotherapeutic drug-induced fibrosis, radiation-induced fibrosis, and injuries and burns.

Fibrotic disorders are often hepatic-related, and there is frequently a nexus between such disorders and the inappropriate accumulation of liver cholesterol and triglycerides within the hepatocytes and Kupffer cells. This accumulation appears to result in a pro-inflammatory response that leads to liver fibrosis and cirrhosis. Hepatic disorders having a fibrotic component include non-alcoholic fatty liver disease (NAFLD) and non-alcoholic steatohepatitis (NASH). NAFLD occurs when steatosis (fat deposition in the liver) is present that is not due to excessive alcohol use. It is related to insulin resistance and the metabolic syndrome. NASH is the most extreme form of NAFLD, and is regarded as a major cause of cirrhosis of the liver of unknown cause.

### Pharmaceutical Compositions

The IL-10 polypeptides of the present disclosure may be in the form of compositions suitable for administration to a subject. In general, such compositions are “pharmaceutical compositions” comprising IL-10 and one or more pharmaceutically acceptable or physiologically acceptable diluents, carriers or excipients. In certain embodiments, the IL-10 polypeptides are present in a therapeutically acceptable amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein. Furthermore, the pharmaceutical compositions may be used in combination with other therapeutically active agents or compounds as described herein in order to treat or prevent the diseases, disorders and conditions as contemplated by the present disclosure.

The pharmaceutical compositions typically comprise a therapeutically effective amount of an IL-10 polypeptide contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments. Any drug delivery apparatus may be used to deliver IL-10, including implants (e.g., implantable pumps) and catheter systems, slow injection pumps and devices, all of which are well known to the skilled artisan. Depot injections, which are generally administered subcutaneously or intramuscularly, may also be utilized to release the polypeptides disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or

oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor ELTM (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The pharmaceutical compositions containing the active ingredient may be in a form suitable for oral use, for example, as tablets, capsules, troches, lozenges, aqueous or oily suspensions, dispersible powders or granules, emulsions, hard or soft capsules, or syrups, solutions, microbeads or elixirs. In particular embodiments, an active ingredient of an agent co-administered with an IL-10 agent described herein is in a form suitable for oral use. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed.

The present disclosure contemplates the administration of the IL-10 polypeptides in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The IL-10 polypeptides contemplated by the present disclosure may be in the form of any other suitable pharmaceutical composition (e.g., sprays for nasal or inhalation use) currently known or developed in the future.

The concentration of a polypeptide or fragment thereof in a formulation can vary widely (e.g., from less than about 0.1%, usually at or at least about 2% to as much as 20% to 50% or more by weight) and will usually be selected primarily based on fluid volumes, viscosities, and subject-based factors in accordance with, for example, the particular mode of administration selected.

### Routes of Administration

The present disclosure contemplates the administration of IL-10 (e.g., IL-10 polypeptide), and compositions thereof, in any appropriate manner. Suitable routes of administration include parenteral (e.g., intramuscular, intravenous, subcutaneous (e.g., injection or implant), intraperitoneal, intracisternal, intraarticular, intraperitoneal, intracerebral (intraparenchymal) and intracerebroventricular), oral, nasal, vaginal, sublingual, intraocular, rectal, topical (e.g., transdermal), sublingual and inhalation. Depot injections, which are generally administered subcutaneously or intramuscularly, may also be utilized to release the IL-10 polypeptides disclosed herein over a defined period of time.

Particular embodiments of the present disclosure contemplate parenteral administration. In some particular embodiments, the parenteral administration is intravenous, and in other particular embodiments the parenteral administration is subcutaneous.

### Combination Therapy

The present disclosure contemplates the use of IL-10 molecules in combination with one or more active therapeutic agents (e.g., cytokines) or other prophylactic or therapeutic modalities (e.g., radiation). In such combination therapy, the various active agents frequently have different, complementary mechanisms of action. Such combination therapy may be especially advantageous by allowing a dose reduction of one or more of the agents, thereby reducing or eliminating the adverse effects associated with one or more of the agents. Furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain embodiments, the IL-10 polypeptides and the one or more active therapeutic agents or other prophylactic or therapeutic modalities are administered or applied sequentially, e.g., where one agent is administered prior to one or more other agents. In other embodiments, the IL-10 polypeptides and the one or more active therapeutic agents or other prophylactic or therapeutic modalities are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

The IL-10 polypeptides of the present disclosure may be used in combination with at least one other (active) agent in any manner appropriate under the circumstances. In one embodiment, treatment with the at least one active agent and at least one IL-10 polypeptide of the present disclosure is maintained over a period of time. In another embodiment, treatment with the at least one active agent is reduced or discontinued (e.g., when the subject is stable), while treatment with the IL-10 polypeptide of the present disclosure is maintained at a constant dosing regimen. In a further embodiment, treatment with the at least one active agent is reduced or discontinued (e.g., when the subject is stable), while treatment with the IL-10 polypeptide of the present disclosure is reduced (e.g., lower dose, less frequent dosing or shorter treatment regimen). In yet another embodiment, treatment with the at least one active agent is reduced or discontinued (e.g., when the subject is stable), and treatment with the IL-10 polypeptide of the present disclosure is increased (e.g., higher dose, more frequent dosing or longer treatment regimen). In yet another embodiment, treatment with the at least one active agent is maintained and treatment with the IL-10 polypeptide of the present disclosure is reduced or discontinued (e.g., lower dose, less frequent dosing or shorter treatment regimen). In yet another embodiment, treatment with the at least one active agent and treatment with the IL-10 polypeptide of the present disclosure are reduced or discontinued (e.g., lower dose, less frequent dosing or shorter treatment regimen).

While particular agents suitable for use in combination with the IL-10 polypeptides (e.g., PEG-IL-10) disclosed herein are set forth hereafter, it is to be understood that the present disclosure is not so limited. Hereafter, certain agents are set forth in specific categories of exemplary diseases, disorders and conditions; however, it is to be understood that there is often overlap between one or more categories (e.g., certain agents may have both cardiovascular and anti-inflammatory effects).

Fibrotic Disorders and Cancer. The present disclosure provides methods for treating and/or preventing a proliferative condition; a fibrotic disease, disorder, or condition; cancer, tumor, or precancerous disease, disorder or condition with an IL-10 molecule and at least one additional therapeutic or diagnostic agent.

Examples of chemotherapeutic agents include, but are not limited to, alkylating agents; alkyl sulfonates; aziridines; ethylenimines and methylamelamines; nitrogen mustards; nitrosureas; antibiotics; folic acid analogs; purine analogs; pyrimidine analogs; androgens; anti-adrenals; folic acid replenishers; hydroxyurea; vindesine; dacarbazine; mannomustine; arabinoside (Ara-C); cyclophosphamide; thiotepa; taxoids, e.g., paclitaxel and doxetaxel; chlorambucil; gemcitabine; 6-thioguanine; mercaptopurine; methotrexate; platinum and platinum coordination complexes; vinblastine; etoposide; ifosfamide; mitomycin C; mitoxantrone; vincristine; vinorelbine; navelbine; novantrone; teniposide; daunomycin; CPT11; topoisomerase inhibitors; capecitabine and anti-hormonal agents; antiandrogens; hormones and related hormonal agents; and pharmaceutically acceptable salts, acids or derivatives of any of the above.

Additional treatment modalities that may be used in combination with the IL-10 polypeptides include a cytokine or cytokine antagonist, such as IL-12, INFα, or anti-epidermal growth factor receptor, radiotherapy, a monoclonal antibody against another tumor antigen, a complex of a monoclonal antibody and toxin, a T-cell adjuvant, bone marrow transplant, or antigen presenting cells (e.g., dendritic cell therapy). Vaccines (e.g., as a soluble protein or as a nucleic acid encoding the protein) are also provided herein.

The present disclosure encompasses pharmaceutically acceptable salts, acids or derivatives of any of the above.

Cholesterol Homeostasis Agents. Particular embodiments of the present disclosure involve combinations of IL-10 polypeptides with agents associated with cholesterol homeostasis. Many of these agents target different pathways involving the absorption, synthesis, transport, storage, catabolism, and excretion of cholesterol, and are thus particularly useful candidates for combination therapy.

Examples of therapeutic agents useful in combination therapy for the treatment of hypercholesterolemia (and thus frequently atherosclerosis, for example) include statins; bile acid resins (sequestrants); ezetimibe (ZETIA); fibric acid (e.g., TRICOR) and fibrates; niacins (e.g., NIACOR); cholesterol absorption inhibitors; fat absorption inhibitors; PCSK9 modulators; and/or a combination of the aforementioned (e.g., VYTORIN (ezetimibe with simvastatin). Alternative cholesterol treatments that may be candidates for use in combination with the IL-10 polypeptides described herein include various supplements and herbs (e.g., garlic, policosanol, and guggul).

The present disclosure encompasses pharmaceutically acceptable salts, acids or derivatives of any of the above.

Immune and Inflammatory Conditions. The present disclosure provides methods for treating and/or preventing immune- and/or inflammatory-related diseases, disorders and conditions, as well as disorders associated therewith, with an IL-10 polypeptide (e.g., PEG-IL-10) and at least one additional agent having immune- and/or inflammatory-related properties. By way of example, an IL-10 polypeptide may be administered with an agent having efficacy in a cardiovascular disorder having an inflammatory component.

Examples of therapeutic agents useful in combination therapy include, but are not limited to, non-steroidal anti-inflammatory drugs; acetic acid derivatives; fenamic acid derivatives; biphenylcarboxylic acid derivatives; oxicams; salicylate; and the pyrazolones. Other combinations include selective cyclooxygenase-2 (COX-2) inhibitors, selective cyclooxygenase 1 (COX 1) inhibitors, and non-selective cyclooxygenase (COX) inhibitors.

Other active agents for combination include steroids such as prednisolone, prednisone, methylprednisolone, betamethasone, dexamethasone, or hydrocortisone. dose required when treating patients in combination with the present IL-10 polypeptides.

Additional examples of active agents for combinations for treating, for example, rheumatoid arthritis include cytokine suppressive anti-inflammatory drug(s) (CSAIDs); antibodies to or antagonists of other human cytokines or growth factors, for example, TNF, LT, IL-1β, IL-2, IL-6, IL-7, IL-8, IL-15, IL-16, IL-18, EMAP-II, GM-CSF, FGF, or PDGF.

Particular combinations of active agents may interfere at different points in the autoimmune and subsequent inflammatory cascade, and include TNF antagonists like chimeric, humanized or human TNF antibodies, REMICADE, anti-TNF antibody fragments (e.g., CDP870), and soluble p55 or p75 TNF receptors, derivatives thereof, p75TNFRIgG (ENBREL.) or p55TNFR1gG (LENERCEPT), soluble IL-13 receptor (sIL-13), and also TNFα converting enzyme (TACE) inhibitors; similarly IL-1 inhibitors (e.g., Interleukin-1-converting enzyme inhibitors) may be effective. Other combinations include Interleukin 11, anti-P7s and p-selectin glycoprotein ligand (PSGL). Other examples of agents useful in combination with the IL-10 polypeptides described herein include interferon-β1a (AVONEX); interferon-β1b (BETASERON); copaxone; hyperbaric oxygen; intravenous immunoglobulin; clabribine; and antibodies to or antagonists of other human cytokines or growth factors (e.g., antibodies to CD40 ligand and CD80).

The present disclosure encompasses pharmaceutically acceptable salts, acids or derivatives of any of the above.

Anti-diabetic and Anti-obesity Agents. Some patients requiring pharmacological treatment for a cholesterol-related disorder(s) are also taking anti-diabetic and/or anti-obesity agents. The present disclosure contemplates combination therapy with numerous anti-diabetic agents (and classes thereof), including 1) insulin, insulin mimetics and agents that entail stimulation of insulin secretion; 2) biguanides and other agents that act by promoting glucose utilization, reducing hepatic glucose production and/or diminishing intestinal glucose output; 3) alpha-glucosidase inhibitors and other agents that slow down carbohydrate digestion and consequently absorption from the gut and reduce postprandial hyperglycemia; 4) thiazolidinediones; 5) glucagon-like-peptides including DPP-IV inhibitors, GLP-1 and GLP-1 agonists and analogs; 6) and DPP-IV-resistant analogues (incretin mimetics), PPAR gamma agonists, dual-acting PPAR agonists, pan-acting PPAR agonists, PTP1B inhibitors, SGLT inhibitors, insulin secretagogues, glycogen synthase kinase-3 inhibitors, immune modulators, beta-3 adrenergic receptor agonists, 1 lbeta-HSD1 inhibitors, amylin analogues; and nuclear receptor binding agents (e.g., a Retinoic Acid Receptor (RAR) binding agent, a Retinoid X Receptor (RXR) binding agent, a Liver X Receptor (LXR) binding agent and a Vitamin D binding agent).

Furthermore, the present disclosure contemplates combination therapy with agents and methods for promoting weight loss, such as agents that stimulate metabolism or decrease appetite, and modified diets and/or exercise regimens to promote weight loss.

The present disclosure encompasses pharmaceutically acceptable salts, acids or derivatives of any of the above.

### Dosing

The IL-10 polypeptides of the present disclosure may be administered to a subject in an amount that is dependent upon, for example, the goal of the administration (e.g., the degree of resolution desired); the age, weight, sex, and health and physical condition of the subject; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (i.e., the maximum tolerated dose, “MTD”) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount is more than the calculated ED50, in other situations the effective amount is less than the calculated ED50, and in still other situations the effective amount is the same as the calculated ED50.

In addition, an effective dose of the IL-10 polypeptides of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

When an IL-10 polypeptide is PEG-IL-10, the amount of PEG-IL-10 necessary to treat a disease, disorder or condition described herein is based on the IL-10 activity of the conjugated protein, which, as indicated above, can be determined by IL-10 activity assays known in the art. By way of example, in the tumor context, suitable IL-10 activity includes, for example, CD8+ T-cell infiltrate into tumor sites, expression of inflammatory cytokines, such as IFN-γ, IL-4, IL-6, IL-10, and RANK-L, from these infiltrating cells, and increased levels of TNF-α or IFN-γ in biological samples.

Like many drugs, intravenous IL-10 administration is associated with a two-compartment kinetic model (see Rachmawati, H. et al. (2004) Pharm. Res. 21(11):2072-78). Plasma drug concentrations decline in a multi-exponential fashion. Immediately after intravenous administration, the drug rapidly distributes throughout an initial space (minimally defined as the plasma volume), and then a slower, equilibrative distribution to extravascular spaces (e.g., certain tissues) occurs. The pharmacokinetics of subcutaneous recombinant hIL-10 has also been studied (Radwanski, E. et al. (1998) Pharm. Res. 15(12):1895-1901). Volume-of-distribution and other pharmacokinetic considerations are pertinent when assessing appropriate IL-10 dosing-related parameters. Moreover, the leveraging of IL-10 pharmacokinetic and dosing principles may prove invaluable to the success of efforts to target IL-10 agents to specific cell types (see, e.g., Rachmawati, H. (May 2007) Drug Met. Dist. 35(5):814-21).

The present disclosure contemplates administration of any dose and dosing regimen that results in the desired therapeutic outcome. By way of example, but not limitation, when the subject is a human, non-pegylated hIL-10 may be administered at a dose greater than 0.5 μg/kg/day, greater than 1.0 μg/kg/day, greater than 2.5 μg/kg/day, greater than 5 μg/kg/day, greater than 7.5 μg/kg, greater than 10.0 μg/kg, greater than 12.5 μg/kg, greater than 15 μg/kg/day, greater than 17.5 μg/kg/day, greater than 20 μg/kg/day, greater than 22.5 μg/kg/day, greater than 25 μg/kg/day, greater than 30 μg/kg/day, or greater than 35 μg/kg/day. In addition, by way of example, but not limitation, when the subject is a human, pegylated hIL-10 comprising a relatively small PEG (e.g., 5 kDa mono- di-PEG-hIL-10) may be administered at a dose greater than 0.5 μg/kg/day, greater than 0.75 μg/kg/day, greater than 1.0 μg/kg/day, greater than 1.25 μg/kg/day, greater than 1.5 μg/kg/day, greater than 1.75 μg/kg/day, greater than 2.0 μg/kg/day, greater than 2.25 μg/kg/day, greater than 2.5 μg/kg/day, greater than 2.75 μg/kg/day, greater than 3.0 μg/kg/day, greater than 3.25 μg/kg/day, greater than 3.5 μg/kg/day, greater than 3.75 μg/kg/day, greater than 4.0 μg/kg/day, greater than 4.25 μg/kg/day, greater than 4.5 μg/kg/day, greater than 4.75 μg/kg/day, or greater than 5.0 μg/kg/day.

The therapeutically effective amount of PEG-IL-10 can range from about 0.01 to about 100 μg protein/kg of body weight/day, from about 0.1 to 20 μg protein/kg of body weight/day, from about 0.5 to 10 μg protein/kg of body weight/day, or from about 1 to 4 μg protein/kg of body weight/day. In some embodiments, PEG-IL-10 is administered by continuous infusion to delivery about 50 to 800 μg protein/kg of body weight/day (e.g., about 1 to 16 μg protein/kg of body weight/day of PEG-IL-10). The infusion rate may be varied based on evaluation of, for example, adverse effects and blood cell counts.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of the disclosed IL-10 polypeptide (e.g., PEG-IL-10) is contained in a “unit dosage form”. The phrase “unit dosage form” refers to physically discrete units, each unit containing a predetermined amount of a IL-10 polypeptide of the present disclosure, either alone or in combination with one or more additional agents, sufficient to produce the desired effect. It will be appreciated that the parameters of a unit dosage form will depend on the particular agent and the effect to be achieved.

### Kits

The present disclosure also contemplates kits comprising IL-10 polypeptides (e.g., PEG-IL-10), and pharmaceutical compositions thereof. The kits are generally in the form of a physical structure housing various components, as described below, and may be utilized, for example, in practicing the methods described above (e.g., administration of an IL-10 polypeptide to a subject in need of restoring cholesterol homeostasis).

A kit can include one or more of the IL-10 polypeptides disclosed herein (provided in, e.g., a sterile container), which may be in the form of a pharmaceutical composition suitable for administration to a subject. The IL-10 polypeptides can be provided in a form that is ready for use or in a form requiring, for example, reconstitution or dilution prior to administration. When the IL-10 polypeptides are in a form that needs to be reconstituted by a user, the kit may also include buffers, pharmaceutically acceptable excipients, and the like, packaged with or separately from the IL-10 polypeptides. When combination therapy is contemplated, the kit may contain the several agents separately or they may already be combined in the kit. Each component of the kit may be enclosed within an individual container, and all of the various containers may be within a single package. A kit of the present disclosure may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); p1=picoliter(s); s or sec=second(s); min=minute(s); h or hr=hour(s); aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; D3=inclusion bodies; HPLC=high performance liquid chromatography; BW=body weight; U=unit; ns=not statistically significant; PBS=phosphate-buffered saline; IHC=immunohistochemistry; EDTA=ethylenediaminetetraacetic acid; SDS-PAGE=sodium dodecyl sulfate polyacrylamide gel electrophoresis; RLU=relative light units; nm=nanometer; LOD=limit of detection; LOQ=limit of quantitation.

### Materials and Methods

The following general materials and methods were used, where indicated, or may be used in the Examples below:

Molecular Biology Procedures. Standard methods in molecular biology are described in the scientific literature (see, e.g., Sambrook and Russell (2001) Molecular Cloning, 3^{rd} ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)).

Antibody-related Processes. Production, purification, and fragmentation of polyclonal and monoclonal antibodies are described (e.g., Harlow and Lane (1999) Using Antibodies, Cold Spring Harbor Laboratory Press, Cold Spring Harbor, N.Y.); standard techniques for characterizing ligand/receptor interactions are available (see, e.g., Coligan et al. (2001) Current Protocols in Immunology, Vol. 4, John Wiley, Inc., NY); methods for flow cytometry, including fluorescence-activated cell sorting (FACS), are available (see, e.g., Shapiro (2003) Practical Flow Cytometry, John Wiley and Sons, Hoboken, N.J.); and fluorescent reagents suitable for modifying nucleic acids, including nucleic acid primers and probes, polypeptides, and antibodies, for use, for example, as diagnostic reagents, are available (Molecular Probes (2003) Catalogue, Molecular Probes, Inc., Eugene, Oreg.; Sigma-Aldrich (2003) Catalogue, St. Louis, Mo.). Further discussion of antibodies appears elsewhere herein.

Software. Software packages and databases for determining, e.g., antigenic fragments, leader sequences, protein folding, functional domains, glycosylation sites, and sequence alignments, are available (see, e.g., GCG Wisconsin Package (Accelrys, Inc., San Diego, Calif.); and DeCypher™ (TimeLogic Corp., Crystal Bay, Nev.).

Pegylation. Pegylated IL-10 as described herein may be synthesized by any means known to the skilled artisan. Exemplary synthetic schemes for producing mono-PEG-IL-10 and a mix of mono-/di-PEG-IL-10 have been described (see, e.g., U.S. Pat. No. 7,052,686; US Pat. Publn. No. 2011/0250163; WO 2010/077853). Particular embodiments of the present disclosure comprise a mix of selectively pegylated mono- and di-PEG-IL-10. In addition to leveraging her own skills in the production and use of PEGs (and other drug delivery technologies) suitable in the practice of the present disclosure, the skilled artisan is familiar with many commercial suppliers of PEG-related technologies (e.g., NOF America Corp (Irvine, Calif.) and Parchem (New Rochelle, N.Y.)).

MC/9 In Vitro Assay. The relative potency (bioactivity) of the IL-10 molecules described herein may be determined using any art-accepted assay or methodology, such as an MC/9 bioassay (see generally, Gomi, K., et al., J. Immuno. 165(11):6545-52 (Dec. 1, 2000)). MC/9 is a murine mast cell line that expresses the endogenous MuIL-10 receptors (R1 and R2). MC/9 cell proliferation occurs in response to stimulation with rMuIL-10 and rHuIL-10. Assay reagents and materials are commercially available from many sources (e.g., R&D Systems, USA; and Cell Signaling Technology, Danvers, Mass.).

In the MC/9 bioassay used herein, 1×10⁴ cells/well were plated and incubated with 3-fold dilutions of rHuIL-10 standards and test samples. Cells were cultured at 37° C., 5% CO₂ for 40-56 hr. After incubation, plates were equilibrated to room temperature for 20-40 min, after which 100 μL of CellTiter GLO (Promega Corp; Madison, Wis.) was added to all wells. Plates where then incubated at room temperature while shaking for 20-40 min, after which they were read on a Luminescence plate reader at a wavelength of 395 nm. For each group, the mean RLU were determined for each concentration. A fit-constrained and independent 4-parameter logistic response curve for each series of samples was generated using the mean RLU vs. log of the concentration. Results were reported relative to the reference potency standard as % relative potency where the reference standard has a potency of 100%. The reported values were generated from the average of at least 3 determinations (e.g., 3 plates).

The protein activity of recombinant hIL-10 may also be assessed by a short-term proliferation bioassay utilizing the MC/9 cell line. Proliferation may be measured by colorimetric means using Alamar Blue, a growth indicator dye, based on detection of metabolic activity. The biological activity of recombinant hIL-10 may be assessed by the EC50 value, or the concentration of protein at which half-maximal stimulation is observed in a dose-response curve.

Exemplary IL-10 Purification Methods Described in the Literature. The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY). Particular methods used, or that may be used, in the methods of the present disclosure are set forth herein.

The scientific and patent literature describes IL-10 purification methods, and such methods are known to the skilled artisan. By way of example, U.S. Pat. No. 5,710,251 describes a method for purifying hIL-10 from a CHO cell line culture medium. Briefly, the method subjects a CHO cell culture supernatant to a series of chromatography steps comprising cation-exchange chromatography (utilizing an SSepharose® column), anion-exchange chromatography (utilizing a Q-Sepharose® column), hydroxyapatite chromatography, and gel-filtration chromatography (utilizing a Sephacryl® column).

In addition, U.S. Pat. No. 5,710,251 describes purification of hIL-10 from E. coli. Briefly, E. coli is transformed with an expression construct such that rhIL-10 is produced intracellularly, and it is present as one component of insoluble inclusion bodies. After fermentation, the inclusion bodies pellets containing IL-10 are isolated from the rest of the cellular material by centrifugation. The inclusion body pellets are then subjected to wash clarification and solubilized to denature protein. Refolding is carried out utilizing a procedure commonly used for proteins having similar properties to IL-10. Thereafter, a series of chromatography steps (similar to those described above for IL-10 purification from a CHO cell line culture medium) are performed: cation-exchange chromatography (utilizing an S-Sepharose® column), anion-exchange chromatography (utilizing a QSepharose® column), hydroxyapatite chromatography, and gel-filtration chromatography (utilizing a Sephacryl® column). As described below, embodiments of the present disclosure comprise modification and optimization of certain of the foregoing steps.

SDS-PAGE Electrophoresis. Protein samples were run on a 12% Bis-Tris gel (Invitrogen) in 1× MES SDS running buffer (Invitrogen) at 200-volt for 37 min. To prepare sample for electrophoresis, 16 μL of refolded material was mixed with 6 μL of 4× LDS sample buffer (Invitrogen) and 2.4 μL of 10× NuPage sample reducing agent (Invitrogen). To prepare unfolded sample for electrophoresis, 1μL of unfolded material was mixed with 15 μL of water, 6 μL of 4× LDS sample buffer and 2.4 μL of 10× NuPage sample reducing agent. After electrophoresis, Simply Blue was used to stain the separated proteins, and an image was captured using GE's ImageQuant LAS 500 imager (GE Healthcare Bio-sciences, Pittsburgh, Pa.). Densitometry was performed using 1 μg, 0.5 μg and 0.25 μg of commercially-available IL-10 as the concentration standard. The procedure followed the manufacturer's protocol.

### EXAMPLE 1

### IL-10 Concentration in Refold Buffer

This example indicates that, in contrast to previously described methods where refolding is volume-dependent and IL-10 concentration is undefined, protein refolding is, in fact, dependent on the IL-10 concentration.

Inclusion bodies were thawed at ambient temperature, and resuspended at a density of 2 g of inclusion bodies per 10 mL of inclusion bodies suspension buffer (50 mM Tris, 4 mM DTT (Acro Biotech; Rancho Cucamonga, Calif.), 7M guanidine, and pH 8.25. Solubilized inclusion bodies were kept at room temperature on a rocking platform for 3-20 hr, and the solubilized material containing IL-10 was separated from the insoluble debris by centrifugation at maximum speed (16000 g) for 15 min at ambient temperature. The supernatant contained unfolded IL-10 in its native state. Prior to initiating the refolding process, 1 μL of the solubilized inclusion bodies suspension was analyzed via SDS-PAGE to determine the purity of the inclusion bodies and the amount of IL-10 in the solubilized material (data not shown). Spectrophotometry was also performed to measure the solubilized material's absorbance at wavelength 260 nm, 280 nm and 320 nm (data not shown).

Following wash clarification, inclusion bodies were solubilized to denature protein, which then underwent a refolding procedure. Briefly, a Dynamax peristaltic pump was used to add the unfolded IL-10 at approximately 1/15^{th} the recirculation rate of the refolding buffer through a 17 cm I.D. tube. The refolding apparatus was used to gradually dilute the guanidine concentration in the unfolded IL-10 from 7 M guanidine to 0.45 M guanidine. Upon addition, the unfolded IL-10 remained at an intermediate guanidine concentration for 6 sec before it was completely added into the bulk refold chamber, which was at the final concentration of 0.45M guanidine. The refolding buffer was recirculated at a rate of 1 volume every 10 min with a Masterflex L/S Easy-Load II pump. The refolding mixture was gently agitated with a stir bar with a speed of ˜6 on a Corning stir plate.

A matrix of experiments was performed, and conditions were evaluated, to determine the optimal IL-10 refolding environment. Briefly, temperatures of 4° C., 25° C. and 37° C. were evaluated; concentration ranges from 0.05 to 10 mg of IL-10/L of refold buffer were assessed; redox potential was evaluated by testing different ratios of oxidized and reduced glutathione in the refolding buffer; and specific ranges (0 mM-2M) of different amino acids were examined to identify the refolding buffer components.

Using the refolding apparatus, a sufficient amount of unfolded IL-10 was serially pulsed-diluted in a refold buffer and redox environment that enriched the proper folding of IL-10. At 350 mL of refold buffer, refolding 1 mg, 4 mg, and 11 mg of denatured IL-10 resulted in the same amount of properly folded material. In addition, it was determined that the addition of unfolded rHuIL-10 monomer at a concentration of 0.15 mg/mL increased the yield of properly-folded dimeric IL-10 from 1.5 to 3 -fold relative to refolding with concentration of 3 mg/mL IL-10 or higher. In particular, when refolding was conducted at a higher IL-10 concentration, the majority of IL-10 was lost as insoluble aggregates, and when refolding was conducted at a lower IL-10 concentration, the final yield of properly folded IL-10 decreased and the downstream processing time increased.

The relationship between the IL-10 concentration in the refolding buffer and total yield is most apparent at cGMP manufacturing scale, shown in Table 1.

TABLE 1

| | IL-10 Conc. in | Recovery | | Yield Efficiency |
|---|---|---|---|---|
| | Refold | From | Final | (% of |
| Lot Number | Buffer (g/mL) | Refold (g) | Yield (g) | IL-10 input) |
| E14-0225 | 0.388 | 97.36 | 2.13 | 1.5 |
| 14-0356 | 0.68 | 127.18 | 3.7 | 1.4 |
| 14-0463 | 0.277 | 254.2 | 8.98 | 3.2 |
| 14-0789 | 0.16 | 300.72 | 7.2 | 4.5 |

As depicted in Table 1, high concentrations of IL-10 in the refolding buffer lead to the poorest yields, whereas concentrations approximating 0.15 mg/mL lead to the greatest percent recoveries. The findings described in this example were also observed at a larger production scale (data not shown).

### EXAMPLE 2

### Addition of Arginine to Refold Buffer

This example indicates that the addition of L-Arginine to refold buffer has a positive effect on the amount of properly folded IL-10 produced.

To facilitate refolding of recombinant proteins obtained from inclusion bodies, 0.1 to 1 M arginine is often used in solvents for refolding proteins by dialysis or dilution (see, e.g., Tsumoto, K. et al., (2004) Biotechnol. Prog. 20:1301-08). However, there is little discussion in the scientific and patent literature regarding the addition of arginine to a refold buffer for use in the production of IL-10. For example, the IL-10 production process disclosed in U.S. Pat No. 5,710,251 does not utilize arginine in refold buffer. When the use of arginine is discussed as a component of a refold buffer for IL-10, it is suggested that 0.5 M L-Arginine and 100 mM urea be used as refold buffer (Arora et al., REFOLD database).

The addition of low concentrations of L-Arginine was found to positively impact IL-10 yield. As indicated in Table 2, the addition of 0.01-0.1 M arginine to a refold buffer containing 0.15 mg/mL unfolded rHuIL-10 led to at least a two-fold increase of properly folded, dimeric IL-10. This concentration of arginine is much less than that reported by Arora et al.

TABLE 2

| | AR19-A1 | AR19-A2 | AR19-A3 | AR19-A4 | AR19-A5 | AR19-A6 |
|---|---|---|---|---|---|---|
| Arginine | 0 | 0.01 | 0.1 | 0.45 | 0.8 | 2 |
| Concentration (M) | | | | | | |
| Final % IL-10 Yield | 49 | 53 | 46 | 33 | 26 | 25 |
| Quantitation of | 0.5 | 2.44 | 1.04 | 0.59 | 0.36 | 1.14 |
| refold IL-10 (mgs) | | | | | | |

Thus, the addition of 0.1 M arginine was observed to be useful in increasing the yield of refolded IL-10 by approximately two-fold over the yield of a refold performed in the absence of Arginine.

### EXAMPLE 3

### Optimization of UFDF Buffer

During the manufacturing process, substantial loss of IL-10 protein was found to occur immediately after the refolding, wherein the mixture of folded and unfolded proteins is concentrated and exchanged into a buffer conducive to purification via an SP Sepharose® column. This step is often termed ultrafiltration/diafiltration (UFDF).

In order to enhance protein solubility and prevent substantial loss of IL-10 due to concentration-dependent precipitation, the impact of the addition of arginine and sodium chloride to the UFDF buffer, or to the buffer into which the refold buffer is exchanged, was assessed. The presence of 0.1 M arginine in the UFDF buffer (20 mM Bis-Tris pH 6.5) was found to increase the yield by an estimated two-fold.

Taken as a whole, the experiments described herein yielded the optimal IL-10 refold conditions, wherein rHuIL-10 concentration is between 0.05 to 0.3 mg/mL, with arginine concentration between 0.01 and 0.1 M. Indeed, the presence of 0.1 M arginine in the refold buffer and in the UFDF buffer consistently increased the total refolded and recovered IL-10 by two-to-four—fold. The final refold environment was optimally maintained at pH 8.3, in the presence of 20% Sucrose (Amesco), 0.1M L-Arginine (Sigma), 50 mM Tris (Corning), 0.45 mM oxidized glutathione (Sigma) and 0.05 mM reduced glutathione (Sigma).

### EXAMPLE 4

### Recovery of IL-10 From a Commercial Manufacturing Process

This example indicates that the amount of refolded IL-10 recovered in a commercial cGMP manufacturing process is influenced by the IL-10 input.

The general methodology described in Example 1 was utilized herein. Briefly, inclusion bodies were solubilized in a suspension buffer, and the solubilized material containing linearized, non-folded IL-10 was separated from the insoluble debris by centrifugation, which resulted in a supernatant containing unfolded IL-10 in its native, unfolded state. Prior to initiating the refolding process, the solubilized inclusion bodies suspension was analyzed via SDS-PAGE to determine the amount of IL-10 in the solubilized material. Spectrophotometry was also performed to measure the solubilized material's absorbance at several wavelengths, including 280 nm. Thereafter, a wash clarifications step was performed, and the inclusion bodies were solubilized to denature protein, which then underwent a refolding procedure.

As indicated in Example 1, during the manufacturing process substantial loss of IL-10 protein generally occurs immediately after the refolding, wherein the mixture of folded and unfolded proteins is concentrated and exchanged into a buffer conducive to purification via an SP Sepharose® column (as noted above, this step may be termed ultrafiltration/diafiltration (UFDF). As previously indicated, optimal IL-10 refold conditions were observed when rHuIL-10 concentration was between 0.05 to 0.3 mg/mL; high concentrations of IL-10 in the refolding buffer lead to the poorer yields due to precipitation of unfolded and aggregated monomeric IL-10.

TABLE 3

| | Lot Number | | | | | |
|---|---|---|---|---|---|---|
| | Combined | | Combined | | | |
| | Refolds 1 | | Refolds 2 | | Combined Refolds 3 | |
| | 15-0540-A | 15-0540-B | 15-0751-A | 15-0751-B | 15-1069-A | 15-1069-B |
| IB Input (Kg) | 4.6 | 5.9 | 7.0 | 6.3 | 7.2 | 7.2 |
| IL-10 Input from | 64.47 | 75.44 | 80.13 | 58.92 | 42.99 | 93.68 |
| IBs (g) | | | | | | |
| UFDF-1 Recovery | 174.63 | 197.66 | 300.79 | 211.91 | 250.3 | 116.06 |
| (g) | | | | | | |
| SP Recovery (g) | 24.34 | 29.67 | 28.3 | 25.97 | 32.93 | 11.54 |

Table 3 sets forth the yield of IL-10 at each step of the commercial manufacturing process. Referring to Table 3, six lots of IL-10 material underwent the steps of unfold, refold, UFDF-1, and purification on an SP Sepharose® column. Each of the six lots underwent the process steps described herein on separate days, and the yield from two of the six lots was combined for further downstream processing; that is, the yields for lot numbers 15-0540-A and 15-0540-B were combined (Combined Refolds 1), the yields for lot numbers 15-0751-A and 15-0751-B were combined (Combined Refolds 2), and the yields for lot numbers 15-1069-A and 15-1069-B were combined (Combined Refolds 3).

In Table 3, “D3 Input” represents the total weight (in kilograms) of washed inclusion bodies; “IL-10 Input from IBs” represents the mass of rHuIL-10 (in grams) obtained from the unfolding step that was added to ˜1000 liters of refolding buffer for the purpose of refolding dimeric rHuIL-10; “UFDF-1” Recovery” represents the mass (in grams) of rHuIL-10 recovered from the first filtration and concentration step; and “SP Recovery” represents the mass (in grams) of rHuIL-10 recovered from the initial capture column.

Referring to lot number 15-0540-A, the 64.47 g obtained from the unfolding step yielded 174.63 g from the refolding step. The putative mass of IL-10 recovered from the refolding step exceeds that from the unfolding step because the putative mass from the refolding step includes non-IL-10 protein from the inclusion bodies and a 280 nm absorbing molecule that gets removed during the SP purification step.

These data illustrate that when the IL-10 input exceeds approximately 80 grams total or approximately 0.09 mg/mL, the recovery substantially diminishes due to precipitation. This result can be illustrated in the last column, wherein the IL-10 input of 93.68 grams yielded an SP recovery (11.54 g) lower than that of any of the other IL-10 input weights. These data are consistent with data described elsewhere herein (e.g., Example 3), wherein optimal IL-10 refold conditions were observed when rHuIL-10 concentration was between 0.05 to 0.3 mg/mL.

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference as if each individual publication or patent application were specifically and individually indicated to be incorporated by reference.

What is claimed is:

1. A method of generating refolded Interleukin-10 (IL-10), comprising:
(a) obtaining a mixture comprising unfolded IL-10 monomers, and (b) contacting the mixture with a refold buffer to produce an admixture comprising refolded IL-10;
wherein the concentration of unfolded IL-10 monomers in the refold buffer is 0.05 g/mL to 0.3 g/mL.

2. The method of claim 1, wherein the concentration of unfolded IL-10 monomers in the refold buffer is 0.1 g/mL to 0.25 g/mL.

3. The method of claim 1, wherein the concentration of unfolded IL-10 monomers in the refold buffer is 0.1 g/mL to 0.2 g/mL.

4. The method of claim 1, wherein the concentration of unfolded IL-10 monomers in the refold buffer is about 0.15 g/mL.

5. The method of any one of claims 1-4, wherein the IL-10 is recombinantly-produced human IL-10 (rhIL-10).

6. The method of claim 5, wherein the rhIL-10 is expressed in bacteria.

7. The method of claim 6, wherein the bacteria is E. coli.

8. The method of any one of the preceding claims, wherein the mixture is produced by combining a plurality of inclusion bodies comprising IL-10 with a suspension buffer.

9. The method of any one of the preceding claims, further comprising adding a redox system to the refold buffer.

10. The method of claim 9, wherein the redox system comprises oxidized and reduced glutathione.

11. The method of any one of the preceding claims, wherein at least one naturally occurring or non-naturally occurring amino acid is added to the refold buffer.

12. The method of claim 11, wherein 0.005 to 0.3 M arginine is added to the refold buffer.

13. The method of claim 11, wherein 0.0075 to 0.25 M arginine is added to the refold buffer.

14. The method of claim 12, wherein 0.05 M to 0.2 M arginine is added to the refold buffer.

15. The method of claim 13, wherein 0.01 M to 0.15 M arginine is added to the refold buffer.

16. The method of claim 14, wherein about 0.1 M arginine and about 0.15 g/mL of unfolded IL-10 monomers are added to the refold buffer.

17. The method of any one of the preceding claims, wherein a wash clarification is performed on the mixture prior to step (b).

18. The method of any one of the preceding claims, wherein an ultrafiltration/diafiltration (UFDF) is performed on the admixture.

19. The method of any one of the preceding claims, wherein the pH of the refold buffer is about pH 8.3.

20. An IL-10 refold buffer, comprising:
(a) a mixture comprising unfolded IL-10 monomers in a concentration of from 0.05 g/mL to 0.3 g/mL; and
(b) arginine in a molarity of from 0.005 to 0.3 M.

21. The refold buffer of claim 20, wherein the concentration of unfolded IL-10 monomers is 0.05 g/mL to 0.25 g/mL.

22. The refold buffer of claim 20, wherein the concentration of unfolded IL-10 monomers is 0.1 g/mL to 0.2 g/mL.

23. The refold buffer of claim 20, wherein the concentration of unfolded IL-10 monomers is about 0.15 g/mL.

24. The refold buffer of any one of claims 20-23, wherein the IL-10 is rhIL-10.

25. The refold buffer of claim 24, wherein the rhIL-10 is expressed in bacteria.

26. The refold buffer of claim 25, wherein the bacteria is E. coli.

27. The refold buffer of any one of claims 20-26, wherein the unfolded IL-10 monomers are obtained from a suspension of inclusion bodies.

28. The refold buffer of any one of claims 20-27, further comprising a redox system.

29. The refold buffer of claim 28, wherein the redox system comprises oxidized glutathione and reduced glutathione.

30. The refold buffer of claim 29, comprising about 0.45 mM oxidized glutathione and about 0.05 mM reduced glutathione.

31. The refold buffer of claim 20, comprising 0.0075 to 0.25 M arginine.

32. The refold buffer of claim 31, comprising 0.05 to 0.2 M arginine.

33. The refold buffer of claim 32, comprising 0.01 to 0.15 M arginine.

34. The refold buffer of claim 33, comprising about 0.1M arginine and about 0.15 g/mL of unfolded IL-10 monomers.

35. The refold buffer of any one of claims 20-34, wherein the mixture is obtained from a wash clarification of a suspension of inclusion bodies comprising IL-10.

36. The refold buffer of any one of claims 20-35, wherein the pH of the refold buffer is about pH 8.3.
